SEQUENCE LISTING (55 sequences)

SEQ ID NO: 1 - protein, 1093 aa, Homo sapiens [sequence omitted]

SEQ ID NO: 2 - DNA, 3282 bp, Homo sapiens
  source 1..3282: mol_type = genomic DNA
atggcgcggccggtccggggagggctcggggccccgcgccgctcgccttgccttctccttctctggctgcttttgcttcggctggagccggtgaccgccgcggccggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccatggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacttgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtcgtgccaccagatgttccaagctacctctcttctcaggggaccctttctgaccgacaagaaaccgtggtcaggaccgagggtggccctcaggccaatgggcacattgagagcaatggtgtgtgtccaagagatgcaagccactttccagagcccgacactcacagcgttgcctgcaggcagccaaagctctgtgctgggtctgcgtatcacaaagagccgtggaaagcgatggagaaagctgaagggacacctgggccacataagatggaacacggtggccgggtcgtatgcagtgactgcaacaccgaagtggactgttactccaggggacaagccttccacccccagcctgtgtccagagacagcgcacagccaagtgcgccaaatggcccggagccgggtgggagtgaccaagagcattctccacatcaccagtgcagcaggactgccgctgggtcctgccccgagtgccaagggtcgctctaccccagtaaccacgatagaatgctgacggctgtgaagaaaaagccaatggcatctctagatgggaaaggggattcttcctggactttagcaaggttgtatcacccggactccacagagctacagcctgcatcttcattaacttcaggcagtccagagcgcgcggaagcccagtacttgcttgtttccaatggccacctccccaaagcatgtgacgccagtcccgagtccacgccactgacaggacagctccccgggaaacagagggtgccactgctgttggcaccaaaaagctag

SEQ ID NO: 3 - protein, 1091 aa, Mus musculus
MARPGPGVLGAPRLAPRLLLWLLLLLLQWPESAGAQAGPRAPCAAACTCAGDSLDCSGRGLATLPRDLPSWTRSLNLSYNRLSEIDSAAFEDLTNLQEVYLNSNELTAIPSLGTASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTVGIFTIAVVCSIVLTSLVWVCIIYQTRKKSEEYSVTNTDETIVPPDVPSYLSSQGTLSDRQETVVRTEGGHQANGHIESNGVCLRDPSLFPEVDIHSTTCRQPKLCVGYTREPWKVTEKADRTAAPHTTAHSGSAVCSDCSTDTAYHPQPVPRDSGQPGTASSQELRQHDREYSPHHPYSGTADGSHTLSGGSLYPSNHDRILPSLKNKAASADGNGDSSWTLAKLHEADCIDLKPSPTLASGSPELMEDAISTEAQHLLVSNGHLPKACDSSPESVPLKGQITGKRRGPLLLAPRS

SEQ ID NO: 4 - protein, 6 aa, synthetic construct
  REGION 1..6: note = Lrig-1 epitope
LNLSYN

SEQ ID NO: 5 - protein, 6 aa, synthetic construct
  REGION 1..6: note = Lrig-1 epitope
LDLNRN

SEQ ID NO: 6 - protein, 6 aa, synthetic construct
  REGION 1..6: note = Lrig-1 epitope
LFLQHN

SEQ ID NO: 7 - protein, 6 aa, synthetic construct
  REGION 1..6: note = Lrig-1 epitope
LNLAGN

SEQ ID NO: 8 - protein, 6 aa, synthetic construct
  REGION 1..6: note = Lrig-1 epitope
LDLSLN

SEQ ID NO: 9 - protein, 6 aa, synthetic construct
  REGION 1..6: note = Lrig-1 epitope
LRLSKN

SEQ ID NO: 10 - protein, 6 aa, synthetic construct
  REGION 1..6: note = Lrig-1 epitope
LKLQRN

SEQ ID NO: 11 - protein, 6 aa, synthetic construct
  REGION 1..6: note = Lrig-1 epitope
LHLEYN

SEQ ID NO: 12 - protein, 6 aa, synthetic construct
  REGION 1..6: note = Lrig-1 epitope
LHLSNN

SEQ ID NO: 13 - protein, 6 aa, synthetic construct
  REGION 1..6: note = Lrig-1 epitope
LVLSFN

SEQ ID NO: 14 - protein, 6 aa, synthetic construct
  REGION 1..6: note = Lrig-1 epitope
LRLSHN

SEQ ID NO: 15 - protein, 6 aa, synthetic construct
  REGION 1..6: note = Lrig-1 epitope
LDLDHN

SEQ ID NO: 16 - protein, 6 aa, synthetic construct
  REGION 1..6: note = Lrig-1 epitope
LTLFGN

SEQ ID NO: 17 - protein, 6 aa, synthetic construct
  REGION 1..6: note = Lrig-1 epitope
LNLGGN

SEQ ID NO: 18 - protein, 12 aa, synthetic construct
  REGION 1..12: note = Lrig-1 epitope
WTRSLNLSYNKL

SEQ ID NO: 19 - protein, 14 aa, synthetic construct
  REGION 1..14: note = Lrig-1 epitope
TEVRNTCFPHGPPI

SEQ ID NO: 20 - protein, 13 aa, synthetic construct
  REGION 1..13: note = Lrig-1 epitope
RLTQLDLNRNRIR

SEQ ID NO: 21 - protein, 15 aa, synthetic construct
  REGION 1..15: note = Lrig-1 epitope
DLNRNRIRLIEGLTF

SEQ ID NO: 22 - protein, 11 aa, synthetic construct
  REGION 1..11: note = Lrig-1 epitope
NSIARIHRKGW

SEQ ID NO: 23 - protein, 14 aa, synthetic construct
  REGION 1..14: note = Lrig-1 epitope
WLPPWLIGRMLQAF

SEQ ID NO: 24 - protein, 11 aa, synthetic construct
  REGION 1..11: note = Lrig-1 epitope
RQVTFGHEGRY

SEQ ID NO: 25 - protein, 15 aa, synthetic construct
  REGION 1..15: note = Lrig-1 epitope
FGHEGRYQCVITNHF

SEQ ID NO: 26 - protein, 15 aa, synthetic construct
  REGION 1..15: note = Lrig-1 epitope
RLTVNVLPSFTKTPH

SEQ ID NO: 27 - protein, 13 aa, synthetic construct
  REGION 1..13: note = Lrig-1 epitope
RRMHVMPDDDVFF

SEQ ID NO: 28 - protein, 15 aa, synthetic construct
  REGION 1..15: note = Lrig-1 epitope
FFITDVKIDDAGVYS

SEQ ID NO: 29 - protein, 13 aa, synthetic construct
  REGION 1..13: note = Lrig-1 epitope
KGDRPLSLTERHH

SEQ ID NO: 30 - protein, 449 aa, synthetic construct
  REGION 1..449: note = A7 heavy chain
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 217 aa, synthetic construct
  REGION 1..217: note = A7 light chain
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 449 aa, synthetic construct
  REGION 1..449: note = C8 heavy chain
EVQLLESGGGLVQPGGSLRLSCAASGFTFSNYYMSWVRQAPGKGLEWVSGISPGDSSTYYADSVKGRFTISRDNSKNTLYLQMNSLRAEDTAVYYCAKGLYSNPNEPFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 217 aa, synthetic construct
  REGION 1..217: note = C8 light chain
QSVLTQPPSASGTPGQRVTISCTGSSSNIGSNYVSWYQQLPGTAPKLLIYDDSQRPSGVPDRFSGSKSGTSASLAISGLRSEDEADYYCGTWDYSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 34 - protein, 455 aa, synthetic construct
  REGION 1..455: note = E7 heavy chain
EVQLLESGGGLVQPGGSLRLSCAASGFTFSSYDMSWVRQAPGKGLEWVSGISPDGSNIYYADSVKGRFTISRDNSKNTLYLQMNSLRAEDTAVYYCAKVGLRCRYEACSYAYGMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 35 - protein, 217 aa, synthetic construct
  REGION 1..217: note = E7 light chain
QSVLTQPPSASGTPGQRVTISCSGSSSNIGSNYVSWYQQLPGTAPKLLIYSDSHRPSGVPDRFSGSKSGTSASLAISGLRSEDEADYYCATWDSSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 36 - protein, 449 aa, synthetic construct
  REGION 1..449: note = G3 heavy chain
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 37 - protein, 217 aa, synthetic construct
  REGION 1..217: note = G3 light chain
QSVLTQPPSASGTPGQRVTISCTGSSSNIGNNNVNWYQQLPGTAPKLLIYSDSHRPSGVPDRFSGSKSGTSASLAISGLRSEDEADYYCGSWDDSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - protein, 455 aa, synthetic construct
  REGION 1..455: note = A8 heavy chain
EVQLLESGGGLVQPGGSLRLSCAASGFTFSDYDMSWVRQVPGKGLEWVSWISHGGGSIYYADSVKGRFTISRDNSKNTLYLQMNSLRAEDTAVYYCARGLGLCKTGLCYYYDAMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 39 - protein, 217 aa, synthetic construct
  REGION 1..217: note = A8 light chain
QSVLTQPPSASGTPGQRVTISCTGSSSNIGNNSVTWYQQLPGTAPKLLIYADNNRPSGVPDRFSGSKSGTSASLAISGLRSEDEADYYCAAWDSSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 40 - protein, 444 aa, synthetic construct
  REGION 1..444: note = B8 heavy chain
EVQLLESGGGLVQPGGSLRLSCAASGFTFSDYYMSWVRQAPGKGLEWVSGISHDSGSKYYADSVKGRFTISRDNSKNTLYLQMNSLRAEDTAVYYCARHWTTFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 41 - protein, 217 aa, synthetic construct
  REGION 1..217: note = B8 light chain
QSVLTQPPSASGTPGQRVTISCSGSSSNIGSNNVTWYQQLPGTAPKLLIYANSNRPSGVPDRFSGSKSGTSASLAISGLRSEDEADYYCGAWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 42 - protein, 455 aa, synthetic construct
  REGION 1..455: note = D9 heavy chain
EVQLLESGGGLVQPGGSLRLSCAASGFTFSNYAMSWVRQAPGKGLEWVSAIYPGGGSIYYADSVKGRFTISRDNSKNTLYLQMNSLRAEDTAVYYCARDILPCPWGRCYYDYAMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 43 - protein, 217 aa, synthetic construct
  REGION 1..217: note = D9 light chain
QSVLTQPPSASGTPGQRVTISCSDSSSNIGSNTVSWYQQLPGTAPKLLIYADNNRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 44 - protein, 455 aa, synthetic construct
  REGION 1..455: note = H6 heavy chain
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 45 - protein, 217 aa, synthetic construct
  REGION 1..217: note = H6 light chain
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - DNA, 24 bp, synthetic construct
  misc_feature 1..24: note = Mouse Lrig-1 forward primer
  source 1..24: mol_type = other DNA
gacggaattcagtgaggagaacct

SEQ ID NO: 47 - DNA, 24 bp, synthetic construct
  misc_feature 1..24: note = Mouse Lrig-1 reverse primer
  source 1..24: mol_type = other DNA
caactggtagtggcagcttgtagg

SEQ ID NO: 48 - DNA, 24 bp, synthetic construct
  misc_feature 1..24: note = Mouse Lrig-2 forward primer
  source 1..24: mol_type = other DNA
tcacaaggaacattgtctgaacca

SEQ ID NO: 49 - DNA, 24 bp, synthetic construct
  misc_feature 1..24: note = Mouse Lrig-2 reverse primer
  source 1..24: mol_type = other DNA
gcctgatctaacacatcctcctca

SEQ ID NO: 50 - DNA, 24 bp, synthetic construct
  misc_feature 1..24: note = Mouse Lrig-3 forward primer
  source 1..24: mol_type = other DNA
cagcaccttgagctgaacagaaac

SEQ ID NO: 51 - DNA, 24 bp, synthetic construct
  misc_feature 1..24: note = Mouse Lrig-3 reverse primer
  source 1..24: mol_type = other DNA
ccagcctttggtaatctcggttag

SEQ ID NO: 52 - DNA, 24 bp, synthetic construct
  misc_feature 1..24: note = Mouse FOXP3 forward primer
  source 1..24: mol_type = other DNA
ctttcacctatcccacccttatcc

SEQ ID NO: 53 - DNA, 24 bp, synthetic construct
  misc_feature 1..24: note = Mouse FOXP3 reverse primer
  source 1..24: mol_type = other DNA
attcatctacggtccacactgctc

SEQ ID NO: 54 - DNA, 20 bp, synthetic construct
  misc_feature 1..20: note = Mouse ACTG1 forward primer
  source 1..20: mol_type = other DNA
ggcgtcatggtgggcatggg

SEQ ID NO: 55 - DNA, 20 bp, synthetic construct
  misc_feature 1..20: note = Mouse ACTG1 reverse primer
  source 1..20: mol_type = other DNA
atggcgtggggaagggcgta

(19) United States
(12) Patent Application Publication
(10) Pub. No.: US 2025/0145708 A1
(43) Pub. Date: May 8, 2025

(54) Epitope of Regulatory T Cell Surface Antigen and Antibody Specifically Binding Thereto

(71) Applicant: GOOD T CELLS, INC., Seoul (KR)

(72) Inventors: Jung Ho Kim, Seoul (KR); Beom Seok Kim, Seoul (KR)

(21) Appl. No.: 19/012,607

(22) Filed: Jan. 7, 2025

Related U.S. Application Data
(62) Division of application No. 17/053,600, filed on Nov. 6, 2020.

(30) Foreign Application Priority Data
May 9, 2018 (KR) 10-2018-0053052

(51) Int. Cl. C07K 16/28; A61P 35/00

(52) U.S. Cl. CPC C07K 16/2803; A61P 35/00; C07K 2317/34

(57) ABSTRACT

The present invention relates to an epitope of leucine-rich and immunoglobulin-like domains 1 (Lrig-1) protein, which is an antigen present on the surface of regulatory T cells, and an antibody or antigen-binding fragment specifically binding thereto.

### CROSS-REFERENCE TO RELATED APPLICATIONS

This application is a divisional of U.S. National Stage application Ser. No. 17/053,600, filed Nov. 2, 2020, of International Patent Application no. PCT/KR2019/005592, filed May 9, 2019, which claims the benefit of priority of Korean Patent Application no. 10-2018-0053052, filed May 9, 2018, each of which is hereby incorporated herein by reference in its entirety.

### REFERENCE TO AN ELECTRONIC SEQUENCE LISTING

This application contains a Sequence Listing which has been submitted electronically and is hereby incorporated by reference in its entirety. The Sequence Listing was created on Jan. 7, 2025, is named “20-1741-WO-US-DIV_ST26.xml” and is 79,145 bytes in size.

### TECHNICAL FIELD

The present invention relates to an epitope of leucine-rich and immunoglobulin-like domains 1 (Lrig-1) protein, which is an antigen present on the surface of regulatory T cells, and an antibody or antigen-binding fragment specifically binding thereto.

### BACKGROUND ART

One of the most important traits in all normal individuals is to have the ability to recognize and eliminate non-self antigens, while not detrimentally responding to antigenic substances that make up the self. As such, non-response of the living body to self antigens is called immunologic unresponsiveness or tolerance. Self-tolerance occurs by eliminating lymphocytes that may have specific receptors for self antigens, or by self-inactivation of the ability to respond after contacting self antigens. In a case where a problem arises in inducing or maintaining self-tolerance, an immune response to self antigens occurs, and the disease resulting therefrom is called autoimmune disease.

For the treatment of the autoimmune disease, a concept of suppressor T cells suggesting the possibility of presence of T cells capable of controlling and suppressing the effector function of conventional T cells was introduced and presented for the first time by Gershon in the early 1970s (R. K. Gershon and K. Kondo, Immunology, 1970, 18:723-37). Since then, studies have been conducted to elucidate biological properties and functions of regulatory T cells in many areas of immunology.

In this connection, it has been reported that the regulatory T cells (Treg cells) play an important role in naturally preventing occurrence of excessive inflammation and immune responses; however, in a case where autoimmune disease and chronic inflammatory disease occur, the function and the number of the regulatory T cells are remarkably decreased. Therefore, in a case of patients with immune and inflammatory diseases, it is important that the regulatory T cells are produced at a normal level, which can be one of the treatments for these diseases.

Until now, studies on genes and proteins which are present specifically in regulatory T cells have been conducted, and it has been presented that substances such as CD25, CTLA4, CD62L, CD38, CD103, GITR, and CD45RB may correspond to marker substances. However, there are no genes and proteins that can target only the regulatory T cells alone.

On the other hand, there are three hypervariable regions called complementarity determining regions (hereinafter referred to as “CDRs”) and four framework regions. The CDRs primarily serve to bind to an epitope on an antigen. The CDRs of each chain are typically referred to as CDR1, CDR2, and CDR3 sequentially starting from the N-terminus, and are also distinguished by the chain where particular CDRs are located.

### Technical Problem

An object of the present invention is to provide an epitope of leucine-rich and immunoglobulin-like domains 1 (Lrig-1) protein present on the surface of regulatory T cells (Treg cells).

Another object of the present invention is to provide an antibody or antigen-binding fragment capable of specifically binding to the epitope.

Yet another object of the present invention is to provide a pharmaceutical composition for preventing or treating cancer, comprising the antibody or antigen-binding fragment capable of specifically binding to the epitope.

Still yet another object of the present invention is to provide a method for preventing or treating cancer, using the antibody or antigen-binding fragment capable of specifically binding to the epitope.

However, the technical problem to be achieved by the present invention is not limited to the above-mentioned problems, and other problems which are not mentioned will be clearly understood by those skilled in the art from the following description.

### Solution to Problem

The present invention relates to an epitope of leucine-rich and immunoglobulin-like domains 1 (Lrig-1) protein, or an antibody or antigen-binding fragment which specifically binds to the epitope.

In the present invention, the “Lrig-1 protein” is a transmembrane protein present on the surface of regulatory T cells, and is composed of leucine-rich repeats (LRRs) and three immunoglobulin-like domains on the extracellular or lumen side, a cell transmembrane sequence, and a cytoplasmic tail portion. The LRIG gene family includes LRIG1, LRIG2, and LRIG3, and the amino acids therebetween are highly conserved. The LRIG1 gene is highly expressed in normal skin and can be expressed in basal and hair follicle cells to regulate proliferation of epithelial stem cells. Therefore, the LRIG1 gene plays an important role in maintaining homeostasis of the epidermis, and its absence may develop psoriasis or skin cancer. It has been reported that in a case where chromosome 3p14.3 portion in which LRIG1 is located is cut off, there is a possibility of developing into cancer cells. In fact, it was identified that expression of LRIG1 is greatly decreased in renal cell carcinoma and cutaneous squamous cell carcinoma. Recently, it has been also found that Lrig-1 is expressed in only about 20 to 30% of cancers. On the other hand, for the purpose of the present invention, the Lrig-1 protein may be, but is not limited to, a protein present in humans or mice.

In an example of the present invention, the Lrig-1 protein may be Lrig-1 protein derived from mammals, including primates such as humans and monkeys, and rodents such as mice and rats.

In an example of the present invention, the Lrig-1 protein may be human-derived Lrig-1 protein represented by SEQ ID NO: 1, which may be encoded by, but is not limited to, a nucleic acid sequence represented by SEQ ID NO: 2 (see Table 1).

TABLE 1

| SEQ ID NO | Sequence information | |
|---|---|---|
| SEQ ID NO: 1 | MARPVRGGLG APRRSPCLLL LWLVLVRLEP VTAAAGPRAP CAAACTCAGD SLDCGGRGLA | 60 |
| | ALPGDLPSWT RSLNLSYNKL SEIDPAGFED LPNLQEVNLS YNKLSEIDPA GFEDLPHLQE | 120 |
| | VYLNNENLTA VPSLGAASSH VVSLFLQHNK IRSVEGSQLK AYLSLEVLDL SLNNITEVRH | 180 |
| | TCFPHGPPIK ELNLAGNRIG TLELGAFDGL SRSLLTLRLS KNRITQLPVR AFKLPRLTQL | 240 |
| | DLNRNRIRLI EGLTFQGLNS LEVLKLQRNN ISKLTDGAFW GLSKMHVLHL EYNSLVEVNS | 300 |
| | GSLYGLTALH QLHLSNNSIA RIHRKGWSFC QKLHELVLSF NNLTRLDEES LAELSSLSVL | 360 |
| | RLSHHSISHI AEGAFKGLRS LRVLDLDHNE ISGTIEDTSG AFSGLDSLSK LNLGGNAIRS | 420 |
| | VQFDAFVKMK NLKELHISSD SFLCDCQLKW LPPWLIGRML QAFVTATCAH PESLKGQSIF | 480 |
| | SVPPESFVCD DFLKPQIITQ PETIMAMVGK DIRFTCSAAS SSSSPMTFAW KKDNEVLTNA | 540 |
| | DMENFVHVHA QDGEVMEYTT ILHLRQVTFG HEGRYQCVIT NHFGSTYSHK ARLTVNVLPS | 600 |
| | FTKTPHDITI RTTTVARLEC AATGHPNPQI AWBKDGGTDF PAARERRMHV MPDDDVFFIT | 660 |
| | DVKIDDAGVY SCTAQNSAGS ISANATLTVL ETPSLVVPLE DRVVSVGETV ALQCKATGNP | 720 |
| | PPRITWFKGD RPLSLTERHH LTPDNQLLVV QNVVAEDAGR YTCEMSNTLG TERAHSQLSV | 780 |
| | LPAAGCRKDG TTVGIFTIAV VSSIVLTSLV WVCIIYQTRK KSEEYSVTNT DETVVPPDVP | 840 |
| | SYLSSQGTLS DRQETVVRTE GGPQANGHIE SNGVCPRDAS HFPEPDTHSV ACRQPKLCAG | 900 |
| | SAYHKEPWKA MEKAEGTPGP HKMEHGGRVV CSDCNTEVDC YSRGQAFHPQ PVSRDSAQPS | 960 |
| | APNGPEPGGS DQEHSPHHQC SRTAAGSCPE CQGSLYPSNH DRMLTAVKKK PMASLDGKGD | 1020 |
| | SSWTLARLYH PDSTELQPAS SLTSGSPERA EAQYLLVSNG HLPKACDASP ESTPLTGQLP | 1080 |
| | GKQRVPLLLA PKS | |
| SEQ ID NO: 2 | atggcgcggc cggtccgggg agggctcggg gccccgcgcc gctcgccttg ccttctcctt | 60 |
| | ctctggctgc ttttgcttcg gctggagccg gtgaccgccg cggccggccc gcgggcgccc | 120 |
| | tgcgcggccg cctgcacttg cgctggggac tcgctggact gcggtgggcg cgggctggct | 180 |
| | gcgttgcccg gggacctgcc ctcctggacg cggagcctaa acctgagtta caacaaactc | 240 |
| | tctgagattg accctgctgg ttttgaggac ttgccgaacc tacaggaagt gtacctcaat | 300 |
| | aataatgagt tgacagcggt accatccctg ggcgctgctt catcacatgt cgtctctctc | 360 |
| | tttctgcagc acaacaagat tcgcagcgtg gaggggagcc agctgaaggc ctacctttcc | 420 |
| | ttagaagtgt tagatctgag tttgaacaac atcacggaag tgcggaacac ctgctttcca | 480 |
| | cacggaccgc ctataaagga gctcaacctg gcaggcaatc ggattggcac cctggagttg | 540 |
| | ggagcatttg atggtctgtc acggtcgctg ctaactcttc gcctgagcaa aaacaggatc | 600 |
| | acccagcttc ctgtaagagc attcaagcta cccaggctga cacaactgga cctcaatcgg | 660 |
| | aacaggattc ggctgataga gggcctcacc ttccaggggc tcaacagctt ggaggtgctg | 720 |
| | aagcttcagc gaaacaacat cagcaaactg acagatgggg ccttctgggg actgtccaag | 780 |
| | atgcatgtgc tgcacctgga gtacaacagc ctggtagaag tgaacagcgg ctcgctctac | 840 |
| | ggcctcacgg ccctgcatca gctccacctc agcaacaatt ccatcgctcg cattcaccgc | 900 |
| | aagggctgga gcttctgcca gaagctgcat gagttggtcc tgtccttcaa caacctgaca | 960 |
| | cggctggacg aggagagcct ggccgagctg agcagcctga gtgtcctgcg tctcagccac | 1020 |
| | aattccatca gccacattgc ggagggtgcc ttcaagggac tcaggagcct gcgagtcttg | 1080 |
| | gatctggacc ataacgagat ttcgggcaca atagaggaca cgagcggcgc cttctcaggg | 1140 |
| | ctcgacagcc tcagcaagct gactctgttt ggaaacaaga tcaagtctgt ggctaagaga | 1200 |
| | gcattctcgg ggctggaagg cctggagcac ctgaaccttg gagggaatgc gatcagatct | 1260 |
| | gtccagtttg atgcctttgt gaagatgaag aatcttaaag agctccatat cagcagcgac | 1320 |
| | agcttcctgt gtgactgcca gctgaagtgg ctgcccccgt ggctaattgg caggatgctg | 1380 |
| | caggcctttg tgacagccac ctgtgcccac ccagaatcac tgaagggtca gagcattttc | 1440 |
| | tctgtgccac cagagagttt cgtgtgcgat gacttcctga agccacagat catcacccag | 1500 |
| | ccagaaacca ccatggctat ggtgggcaag gacatccggt ttacatgctc agcagccagc | 1560 |
| | agcagcagct cccccatgac ctttgcctgg aagaaagaca atgaagtcct gaccaatgca | 1620 |
| | gacatggaga actttgtcca cgtccacgcg cagggcgggg aagtgatgga gtacaccacc | 1680 |
| | atcctgcacc tccgtcaggt cactttcggg cacgagggcc gctaccaatg tgtcatcacc | 1740 |
| | aaccactttg gctccaccta ttcacataag gccaggctca ccgtgaatgt gttgccatca | 1800 |
| | ttcaccaaaa cgccccacga cataaccatc cggaccacca ccatggcccg cctcgaatgt | 1860 |
| | gctgccacag gtcacccaaa ccctcagatt gcctggcaga aggatggagg cacggatttc | 1920 |
| | cccgctgccc gtgagcgacg catgcatgtc atgccggatg acgacgtgtt tttcatcact | 1980 |
| | gatgtgaaaa tagatgacgc aggggtttac agctgtactg ctcagaactc agccggttct | 2040 |
| | atttcagcta atgccaccct gactgtccta gagaccccat ccttggtggt ccccttggaa | 2100 |
| | gaccgtgtgg tatctgtggg agaaacagtg gccctccaat gcaaagccac ggggaaccct | 2160 |
| | ccgccccgca tcacctggtt caagggggac cgcccgctga gcctcactga gcggcaccac | 2220 |
| | ttgacccctg acaaccagct cctggtggtt cagaacgtgg tggcagagga tgcgggccga | 2280 |
| | tatacctgtg agatgtccaa caccctgggc acggagcgag ctcacagcca gctgagcgtc | 2340 |
| | ctgcccgcag caggctgcag gaaggatggg accacggtag gcatcttcac cattgctgtc | 2400 |
| | gtgagcagca tcgtcctgac gtcactggtc tgggtgtgca tcatctacca gaccaggaag | 2460 |
| | aagagtgaag agtacagtgt caccaacaca gatgaaaccg tcgtgccacc agatgttcca | 2520 |
| | agctacctct cttctcaggg gaccctttct gaccgacaag aaaccgtggt caggaccgag | 2580 |
| | ggtggccctc aggccaatgg gcacattgag agcaatggtg tgtgtccaag agatgcaagc | 2640 |
| | cactttccag agcccgacac tcacagcgtt gcctgcaggc agccaaagct ctgtgctggg | 2700 |
| | tctgcgtatc acaaagagcc gtggaaagcg atggagaaag ctgaagggac acctgggcca | 2760 |
| | cataagatgg aacacggtgg ccgggtcgta tgcagtgact gcaacaccga agtggactgt | 2820 |
| | tactccaggg gacaagcctt ccacccccag cctgtgtcca gagacagcgc acagccaagt | 2880 |
| | gcgccaaatg gcccggagcc gggtgggagt gaccaagagc attctccaca tcaccagtgc | 2940 |
| | agcaggactg ccgctgggtc ctgccccgag tgccaagggt cgctctaccc cagtaaccac | 3000 |
| | gatagaatgc tgacggctgt gaagaaaaag ccaatggcat ctctagatgg gaaaggggat | 3060 |
| | tcttcctgga ctttagcaag gttgtatcac ccggactcca cagagctaca gcctgcatct | 3120 |
| | tcattaactt caggcagtcc agagcgcgcg gaagcccagt acttgcttgt ttccaatggc | 3180 |
| | cacctcccca aagcatgtga cgccagtccc gagtccacgc cactgacagg acagctcccc | 3240 |
| | gggaaacaga gggtgccact gctgttggca ccaaaaagct ag | |

In another example of the present invention, the Lrig-1 protein may be, but is not limited to, mouse-derived Lrig-1 protein represented by SEQ ID NO: 3 (see Table 2).

TABLE 2

| SEQ ID NO | Sequence information | |
|---|---|---|
| SEQ ID NO: 3 | MARPGPGVLG APRLAPRLLL WLLLLLLQWP ESAGAQAGPR APCAAACTCA GDSLDCSGRG | 60 |
| | LATLPRDLPS WTRSLNLSYN RLSEIDSAAF EDLTNLQEVY LNSNELTAIP SLGTASIGVV | 120 |
| | SLFLQHNKIL SVDGSQLKSY LSLEVLDLSS NNITEIRSSC FPNGLRIREL NLASNRISIL | 180 |
| | ESGAFDGLSR SLLTLRLSKN RITQLPVKAF KLPRLTQLDL NRNRIRLIEG LTFQGLDSLE | 240 |
| | VLRLQRNNIS RLTDGAFWGL SKMHVLHLEY NSLVEVNSGS LYGLTALHQL HLSNNSISRI | 300 |
| | QRDGWSFCQK LHELILSFNN LTRLDEESLA ELSSLSILRL SHNAISHIAE GAFKGLKSLR | 360 |
| | VLDLDHNEIS GTIEDTSGAF TGLDNLSKLT LFGNKIKSVA KRAFSGLESL EHLNLGENAI | 420 |
| | RSVQFDAFAK MKNLKELYIS SESFLCDCQL KWLPPWLMGR MLQAFVTATC AHPESLKGQS | 480 |
| | IFSVLPDSFV CDDFPKPQII TQPETTMAVV GKDIRFTCSA ASSSSSPMTF AWKKDNEVLA | 540 |
| | NADMENFAHV RAQDGEVMEY TTILHLRHVT FGHEGRYQCI ITNHFGSTYS HKARLTVNVL | 600 |
| | PSFTKIPHDI AIRTGTTARL ECAATGHPNP QIAWQKDGGT DFPAARERRM HVMPDDDVFF | 660 |
| | ITDVKIDDMG VYSCTAQNSA GSVSANATLT VLETPSLAVP LEDRVVTVGE TVAFQCKATG | 720 |
| | SPTPRITWLK GGRPLSLTER HHFTPGNQLL VVQNVMIDDA GRYTCEMSNP LGTERAHSQL | 780 |
| | SILPTPGCRK DGTTVGIFTI AVVCSIVLTS LVWVCIIYQT RKKSEEYSVT NTDETIVPPD | 840 |
| | VPSYLSSQGT LSDRQETVVR TEGGHQANGH IESNGVCLRD PSLFPEVDIH STTCRQPKLC | 900 |
| | VGYTREPWKV TEKADRTAAP HTTAHSGSAV CSDCSTDTAY HPQPVPRDSG QPGTASSQEL | 960 |
| | RQHDREYSPH HPYSGTADGS HTLSGGSLYP SNHDRILPSL KNKAASADGN GDSSWTLAKL | 1020 |
| | HEADCIDLKP SPTLASGSPE LMEDAISTEA QHLLVSNGHL PKACDSSPES VPLKGQITGK | 1080 |
| | RRGPLLLAPR S | |

According to an embodiment of the present invention, as the epitope of the Lrig-1 protein, there is provided an epitope including a polypeptide that consists of an amino acid sequence represented by Formula 1,

Lx¹Lx²x³N. [Formula 1]

In Formula 1, x¹ to x³ may each independently be a neutral amino acid, an acidic amino acid, a basic amino acid, or an aromatic amino acid. Here, the neutral amino acid may be glycine (G), alanine (A), valine (V), leucine (L), isoleucine (I), serine (S), or threonine (T); the acidic amino acid may be aspartic acid (D), glutamic acid (E), asparagine (N), or glutamine (Q); the basic amino acid may be lysine (K), arginine (R), or histidine (H); and the aromatic amino acid may be phenylalanine (F) or tyrosine (Y).

In an example of the present invention, x¹ to x³ may each independently be an amino acid selected from the group consisting of asparagine (N), aspartic acid (D), serine (S), tyrosine (Y), arginine (R), phenylalanine (F), lysine (K), histidine (H), leucine (L), valine (V), threonine (T), alanine (A), glutamine (Q), glutamic acid (E), and glycine (G).

In another example of the present invention, x¹ may be an amino acid selected from the group consisting of asparagine (N), phenylalanine (F), aspartic acid (D), lysine (K), histidine (H), valine (V), arginine (R), and threonine (T); x² may be an amino acid selected from the group consisting of serine (S), glutamine (Q), alanine (A), asparagine (N), glutamic acid (E), aspartic acid (D), phenylalanine (F), and glycine (G); and x³ may be an amino acid selected from the group consisting of tyrosine (Y), histidine (H), glycine (G), arginine (R), asparagine (N), leucine (L), lysine (K), and phenylalanine (F).

In yet another example of the present invention, x¹ to x³ may each independently be an amino acid selected from the group consisting of asparagine (N), aspartic acid (D), serine (S), tyrosine (Y), and arginine (R).

In still yet another example of the present invention, x¹ may be asparagine (N) or aspartic acid (D); x² may be serine (S) or asparagine (N); and x³ may be tyrosine (Y) or arginine (R).

In the present invention, the epitope includes a polypeptide consisting of an amino acid sequence represented by Formula 1, and may consist of 10- to 20-mer, preferably 10- to 15-mer, and more preferably 11- to 14-mer.

In addition, in the present invention, the polypeptide consisting of the amino acid sequence represented by Formula 1 in the epitope may be located at positions 3 to 6, preferably positions 4 to 6, and more preferably position 5 from the N-terminus of the epitope.

As an example of the present invention, the polypeptide consisting of the amino acid sequence represented by Formula 1 may be represented by, but is not limited to, any one amino acid sequence of SEQ ID NOs: 4 to 17 in Table 3 below:

TABLE 3

| | Sequence |
|---|---|
| SEQ ID NO | information |
| SEQ ID NO: 4 | LNLSYN |
| SEQ ID NO: 5 | LDLNRN |
| SEQ ID NO: 6 | LFLQHN |
| SEQ ID NO: 7 | LNLAGN |
| SEQ ID NO: 8 | LDLSLN |
| SEQ ID NO: 9 | LRLSKN |
| SEQ ID NO: 10 | LKLQRN |
| SEQ ID NO: 11 | LHLEYN |
| SEQ ID NO: 12 | LHLSNN |
| SEQ ID NO: 13 | LVLSFN |
| SEQ ID NO: 14 | LRLSHN |
| SEQ ID NO: 15 | LDLDHN |
| SEQ ID NO: 16 | LTLFGN |
| SEQ ID NO: 17 | LNLGGN |

As another example of the present invention, the epitope may be represented by, but is not limited to, an amino acid sequence of SEQ ID NO: 18 or 20.

According to another embodiment of the present invention, as the epitope of the Lrig-1 protein, there is provided an epitope including a polypeptide represented by any one amino acid sequence of SEQ ID NOs: 18 to 29 in Table 4 below:

TABLE 4

| SEQ ID NO | Sequence information |
|---|---|
| SEQ ID NO: 18 | WTRSLNLSYNKL |
| SEQ ID NO: 19 | TEVRNTCFPHGPPI |
| SEQ ID NO: 20 | RLTQLDLNRNRIR |
| SEQ ID NO: 21 | DLNRNRIRLIEGLTF |
| SEQ ID NO: 22 | NSIARIHRKGW |
| SEQ ID NO: 23 | WLPPWLIGRMLQAF |
| SEQ ID NO: 24 | RQVTFGHEGRY |
| SEQ ID NO: 25 | FGHEGRYQCVITNHF |
| SEQ ID NO: 26 | RLTVNVLPSFTKTPH |
| SEQ ID NO: 27 | RRMHVMPDDDVFF |
| SEQ ID NO: 28 | FFITDVKIDDAGVYS |
| SEQ ID NO: 29 | KGDRPLSLTERHH |

According to yet another embodiment of the present invention, there is provided a nucleic acid molecule encoding the epitope provided by the present invention.

The nucleic acid molecule of the present invention includes all nucleic acid molecules obtained by translating the amino acid sequences of the polypeptides provided by the present invention to polynucleotide sequences, as known to those skilled in the art. Therefore, various polynucleotide sequences may be prepared by an open reading frame (ORF), and all of these polynucleotide sequences are also included in the nucleic acid molecule of the present invention.

According to still yet another embodiment of the present invention, there is provided an expression vector into which the isolated nucleic acid molecule provided by the present invention is inserted.

In the present invention, the “vector” is a nucleic acid molecule capable of transporting another nucleic acid linked thereto. One type of vector is a “plasmid,” which refers to circular double-stranded DNA into which an additional DNA segment can be ligated. Another type of vector is a phage vector. Yet another type of vector is a viral vector, where an additional DNA segment can be ligated into the viral genome. Certain vectors are capable of autonomous replication in a host cell into which they are introduced (for example, bacterial vectors having a bacterial origin of replication are episomal mammalian vectors). Other vectors (for example, non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, “plasmid” and “vector” may be used interchangeably as the plasmid is the most commonly used form of vector.

Specific examples of the expression vector in the present invention may be selected from, but are not limited to, the group consisting of commercially widely used pCDNA vectors, F, R1, RP1, Col, pBR322, ToL, Ti vectors; cosmids; phages such as lambda, lambdoid, M13, Mu, p1 P22, Quu, T-even, T2, T3, T7; plant viruses. Any expression vector known, to those skilled in the art, as expression vectors can be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation. However, the present invention is not limited thereto, and those skilled in the art may adopt and use an introduction method appropriate for the expression vector and the host cell which are used. The vector may preferably contain at least one selection marker. However, the present invention is not limited thereto, and selection can be made using the vector that contains no selection marker, depending on whether or not a product is produced. The selection marker is selected depending on the target host cell, which is done using methods already known to those skilled in the art, and thus the present invention has no limitation thereon.

In order to facilitate purification of the nucleic acid molecule of the present invention, a tag sequence may be inserted into and fused to an expression vector. The tag includes, but is not limited to, hexa-histidine tag, hemagglutinin tag, myc tag, or flag tag, and any tag known to those skilled in the art which facilitates purification can be used in the present invention.

According to still yet another embodiment of the present invention, there is provided a host cell line, transfected with the expression vector provided by the present invention.

In the present invention, the “host cell” includes individual cells or cell cultures which may be or have been recipients of the vector(s) for incorporation of a polypeptide insert. The host cell includes progeny of a single host cell, and the progeny may not necessarily be completely identical (in morphology or in genomic DNA complement) to the original parent cell due to natural, accidental, or intentional mutation. The host cell includes cells transfected in vivo with the polynucleotide(s) herein.

In the present invention, the host cell may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as CHO (Chinese hamster ovary cells), SP2/0 (mouse myeloma), human lymphoblastoid, COS, NSO (mouse myeloma), 293T, Bowes melanoma cells, HT-1080, BHK (baby hamster kidney cells), HEK (human embryonic kidney cells), or PERC.6 (human retinal cells); or plant cells. However, the host cell is not limited thereto, and any cell known to those skilled in the art which can be used as a host cell line is available.

According to still yet another embodiment of the present invention, there is provided an antibody or antigen-binding fragment which specifically binds to the epitope of the present invention.

In addition, the antibody according to the present invention specifically binds to an epitope including a polypeptide consisting of an amino acid sequence represented by Formula 1, or an epitope including a polypeptide represented by any one amino acid sequence of SEQ ID NOs: 18 to 29, in Lrig-1 protein present on regulatory T cells, so that the regulatory T cells' function can be suppressed and effector T cells' activity can be maintained or increased, thereby effectively suppressing growth of cancer cells, in particular, solid cancer cells.

As used herein, the “cancer” refers to or indicates a physiological condition characterized by cell growth in mammals which is not regulated in a typical manner. The cancer to be prevented, ameliorated, or treated in the present invention may be solid tumor formed of agglomerates caused by abnormal growth of cells in a solid organ, and may be, but is not limited to, gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, lung cancer, or the like, depending on location of the solid organ.

In the present invention, the antibody as a full-length antibody or as a part of the antibody has the ability to bind to the Lrig-1 protein, and includes any antibody fragment that binds to the Lrig-1 antigen determining site in a competitive manner with the binding molecule of the present invention.

As used herein, the “antibody” refers to a protein molecule which serves as a receptor that specifically recognizes an antigen, including an immunoglobulin molecule that is immunologically reactive with a particular antigen. For the purpose of the present invention, the antigen may be Lrig-1 protein present on the surface of regulatory T cells. Preferably, the antibody may specifically recognize the leucine-rich region or immunoglobulin-like domain of the Lrig-1 protein, but is not limited thereto.

In the present invention, the “immunoglobulin” has a heavy chain and a light chain, and each of the heavy chain and the light chain comprises a constant region and a variable region. The variable region of each of the light chain and the heavy chain contains three hypervariable regions called complementarity determining regions (hereinafter referred to as “CDRs”) and four framework regions. The CDRs primarily serve to bind to an epitope on an antigen. The CDRs of each chain are typically referred to as CDR1, CDR2, and CDR3 sequentially starting from the N-terminus, and are also distinguished by the chain where particular CDRs are located.

In the present invention, the “full-length antibody” has a structure with two full-length light chains and two full-length heavy chains in which each light chain is linked to a heavy chain by disulfide bond, and includes IgA, IgD, IgE, IgM, and IgG. The IgG includes, as subtypes thereof, IgG1, IgG2, IgG3, and IgG4.

In addition, as used herein, the “antigen-binding fragment” refers to a fragment having an antigen-binding function, and examples of the antigen-binding fragment include (i) a Fab fragment consisting of a light chain variable region (VL), a heavy chain variable region (VH), a light chain constant region (CL), and a heavy chain constant region 1 (CH1); (ii) a Fd fragment consisting of VH and CH1 domains; (iii) a Fv fragment consisting of VL and VH domains of a single arm of an antibody; (iv) a dAb fragment (Ward, E. S. et al., Nature 341: 544-546 (1989)) consisting of a VH domain; (v) an isolated CDR region; (vi) a F(ab′)₂ fragment, which is a bivalent fragment including two linked Fab fragments; (vii) a single-chain Fv molecule (scFv), in which a VH domain and a VL domain are linked by a peptide linker that allows the two domains to associate to form an antigen binding site; (viii) a bispecific single-chain Fv dimer; and (ix) a diabody, which is a multivalent or multispecific fragment constructed by gene fusion. The antigen-binding fragment may be obtained as a Fab or F(ab′)₂ fragment in a case where a proteolytic enzyme, for example, papain or pepsin is used, and may be produced through a genetic recombinant technique.

In addition, in the present invention, the antibody may be, but is not limited to, a monoclonal antibody, a polyclonal antibody, a chimeric antibody, a humanized antibody, a bivalent, bispecific molecule, a minibody, a domain antibody, a bispecific antibody, an antibody mimetic, a diabody, a triabody, or a tetrabody, or a fragment thereof.

In addition, as used herein, the “monoclonal antibody” refers to an antibody molecule of a single molecular composition which is obtained from substantially the same antibody population, and exhibits single binding specificity and affinity for a particular epitope.

In the present invention, the “chimeric antibody” is an antibody which is obtained by recombination of a variable region of a mouse antibody and a constant region of a human antibody, and has a greatly improved immune response as compared with the mouse antibody.

In addition, as used herein, the “humanized antibody” refers to an antibody obtained by modifying a protein sequence of an antibody derived from a non-human species so that the protein sequence is similar to an antibody variant naturally produced in humans. For example, the humanized antibody may be prepared as follows. Mouse-derived CDRs may be recombined with a human antibody-derived FR to prepare a humanized variable region, and the humanized variable region may be recombined with a constant region of a preferred human antibody to prepare a humanized antibody.

As used herein, the “binding” or “specific binding” refers to affinity of the antibody or antibody composition herein for an antigen. In antigen-antibody binding, the “specific binding” is distinguishable from non-specific background binding, typically in a case where a dissociation constant (Kd) is less than 1′10⁻⁵ M, less than 1′10⁻⁶ M, or less than 1′10⁻⁷ M. Specific binding can be detected by methods known in the art, such as ELISA, surface plasmon resonance (SPR), immunoprecipitation, and coprecipitation, which include an appropriate control that can distinguish between non-specific binding and specific binding.

The antibody or antigen-binding fragment of the present invention may exist as a multimer, such as a dimer, a trimer, a tetramer, or a pentamer, which includes at least part of antigen-binding capacity of a monomer. Such a multimer also includes a homomultimer or a heteromultimer. Antibody multimers contain a large number of antigen-binding sites, and thus have superior antigen-binding capacity as compared with monomers. Antibody multimers are also easily used to produce multifunctional (that is, bifunctional, trifunctional, tetrafunctional, or the like) antibodies.

As used herein, the “multifunctional” refers to an antibody or antigen-binding fragment which has two or more activities or functions (for example, antigen-binding capacity, enzyme activity, and ligand-or receptor-binding capacity). For example, the antibody of the present invention may be bound to a polypeptide having enzymatic activity, such as luciferase, acetyltransferase, and galactosidase, and the like. Multifunctional antibodies also include multivalent or multispecific (that is, bispecific, trispecific, or the like) forms of antibodies.

According to still yet another embodiment of the present invention, there is provided an antibody-drug conjugate (ADC) comprising the antibody or antigen-binding fragment provided by the present invention and a drug.

As used herein, the “antibody-drug conjugate (ADC)” refers to a form in which the drug and the antibody are chemically linked to each other without degrading biological activity of the antibody and the drug. In the present invention, the antibody-drug conjugate denotes a form in which the drug is bound to an amino acid residue at the N-terminus of the heavy and/or light chain of the antibody, specifically, a form in which the drug is bound to an a-amine group at the N-terminus of the heavy and/or light chain of the antibody.

As used herein, the “drug” may mean any substance having a certain biological activity for a cell, which is a concept including DNA, RNA, or a peptide. The drug may be in a form which contains a reactive group capable of reacting and crosslinking with an α-amine group, and also includes a form which contains a reactive group capable of reacting and crosslinking with an α-amine group and to which a linker is linked.

In the present invention, examples of the reactive group capable of reacting and crosslinking with the a-amine group are not particularly limited in terms of type as long as the reactive group can react and crosslink with an a-amine group at the N-terminus of a heavy or light chain of an antibody. The reactive group includes all types of groups known in the art which react with an amine group. The reactive group may, for example, be any one of isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester, but is not limited thereto.

In the present invention, the antibody-drug conjugate includes, as the antibody or antigen-binding fragment, an antibody or antigen-binding fragment which specifically binds to the epitope of the present invention, that is, an epitope including a polypeptide consisting of an amino acid sequence represented by Formula 1, or an epitope including a polypeptide represented by any one amino acid sequence of SEQ ID NOs: 18 to 29, in Lrig-1 protein, in which the drug may be a drug that can treat cancer, a disease targeted by a Lrig-1 antibody, that is, an anticancer agent.

In the present invention, the anticancer agent may include any drug without limitation as long as the drug is used for prevention, amelioration, or treatment of cancer. The anticancer agent may be, for example, selected from the group consisting of nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, capecitabine, decitabine, mercaptopurine, thioguanine, cladribine, carmofur, raltitrexed, docetaxel, paclitaxel, irinotecan, belotecan, topotecan, vinorelbine, etoposide, vinblastine, idarubicin, mitomycin, bleomycin, dactinomycin, pirarubicin, aclarubicin, peplomycin, temsirolimus, temozolomide, busulfan, ifosfamide, cyclophosphamide, melphalan, altretamine, dacarbazine, thiotepa, nimustine, chlorambucil, mitolactol, leucovorin, tretinoin, exemestane, aminogluthetimide, anagrelide, olaparib, navelbine, fadrozole, tamoxifen, toremifene, testolactone, anastrozole, letrozole, vorozole, bicalutamide, lomustine, 5FU, vorinostat, entinostat, and carmustine. However, the anticancer agent is not limited thereto.

In the present invention, the cancer may be solid tumor formed of agglomerates caused by abnormal growth of cells in a solid organ, and specific examples thereof may include, but are not limited to, gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, lung cancer, or the like, depending on location of the solid organ.

According to still yet another embodiment of the present invention, there is provided a pharmaceutical composition for preventing or treating cancer, comprising, as an active ingredient, the antibody or antigen-binding fragment provided by the present invention, or the antibody-drug conjugate (ADC) provided by the present invention.

In the present invention, the antibody or antigen-binding fragment, or the antibody-drug conjugate obtained by binding a drug thereto, which is contained as an active ingredient in the pharmaceutical composition, specifically binds to an epitope including a polypeptide consisting of an amino acid sequence represented by Formula 1, or an epitope including a polypeptide represented by any one amino acid sequence of SEQ ID NOs: 18 to 29, in Lrig-1 protein present on regulatory T cells, so that the regulatory T cells' function can be suppressed and effector T cells' activity can be maintained or increased, thereby effectively suppressing growth of cancer cells, in particular, solid cancer cells.

In the present invention, the cancer may be solid tumor formed of agglomerates caused by abnormal growth of cells in a solid organ, and specific examples thereof may include, but are not limited to, gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, lung cancer, or the like, depending on location of the solid organ.

Meanwhile, in the present invention, the “prevention” may include, without limitation, any act of blocking symptoms of a disease, or suppressing or delaying the symptoms, using the pharmaceutical composition of the present invention.

In addition, in the present invention, the “treatment” may include, without limitation, any act of ameliorating or beneficially altering symptoms of a disease, using the pharmaceutical composition of the present invention.

In the present invention, the pharmaceutical composition may be characterized by being in the form of capsules, tablets, granules, injections, ointments, powders, or beverages, and the pharmaceutical composition may be characterized by being targeted to humans.

In the present invention, the pharmaceutical composition may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, diluents, or excipients suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition of the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the present invention in any way. The pharmaceutical composition according to the present invention may be formulated in the form of pills, sugar-coated tablets, capsules, liquids, gels, syrups, slurries, or suspensions.

According to still yet another embodiment of the present invention, there is provided a method for preventing or treating cancer, comprising a step of administering, to an individual, the antibody or antigen-binding fragment according to the present invention, or the antibody-drug conjugate (ADC) according to the present invention.

The antibody or antigen-binding fragment of the present invention, and the antibody-drug conjugate of the present invention specifically bind to an epitope including a polypeptide consisting of an amino acid sequence represented by Formula 1, or an epitope including a polypeptide represented by any one amino acid sequence of SEQ ID NOs: 18 to 29, in Lrig-1 protein present on regulatory T cells, so that the regulatory T cells' function can be suppressed and effector T cells' activity can be maintained or increased, thereby effectively suppressing growth of cancer cells, in particular, solid cancer cells.

In the present invention, the “individual” is an individual suspected of developing cancer, and the individual suspected of developing cancer means a mammal, such as humans, mice, and domestic animals, who has developed or is likely to develop the disease in question. However, any individual, who is treatable with the antibody or antibody-drug conjugate of the present invention, is included therein without limitation.

The method of the present invention may comprise administering the antibody or the antibody-drug conjugate in a pharmaceutically effective amount. An appropriate total daily amount used may be determined by an attending physician or veterinarian within the scope of sound medical judgment, and administration may be made once or several times. However, for the purposes of the present invention, a specific therapeutically effective amount for a particular patient is preferably applied differently depending on various factors, including type and degree of reaction to be achieved, the specific composition including whether other agents are used therewith as the case may be, the patient's age, body weight, general health status, sex, and diet, time of administration, route of administration, secretion rate of the composition, duration of treatment, and drugs used simultaneously or in combination with the specific composition, and similar factors well known in the medical field.

Meanwhile, the method for preventing or treating cancer may be, but is not limited to, a combination therapy that further comprises administering a compound or substance having therapeutic activity against one or more cancer diseases.

In the present invention, the “combination” should be understood to represent simultaneous, individual, or sequential administration. In a case where the administration is made in a sequential or individual manner, the second component should be administered at intervals such that beneficial effects of the combination are not lost.

In the present invention, a dosage of the antibody or antibody-drug conjugate may be, but is not limited to, about 0.0001 μg to 500 mg per kg of patient's body weight.

In the present invention, the cancer may be solid tumor formed of agglomerates caused by abnormal growth of cells in a solid organ, and specific examples thereof may include, but are not limited to, gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, lung cancer, or the like, depending on location of the solid organ.

### Advantageous Effects of Invention

The antibody or antigen-binding fragment which specifically binds to an epitope of Lrig-1 protein, according to the present invention, specifically binds to the epitope of the present invention present on regulatory T cells, so that the regulatory T cells' function can be suppressed and effector T cells' activity can be maintained or increased, thereby effectively suppressing growth of cancer cells, in particular, solid cancer cells.

### BRIEF DESCRIPTION OF DRAWINGS

FIG. 1 illustrates a structure of the Lrig-1 protein according to an embodiment of the present invention.

FIG. 2 illustrates a structure of the Lrig-1 protein according to an embodiment of the present invention.

FIG. 3 illustrates an expression level of Lrig-1 mRNA according to an embodiment of the present invention.

FIG. 4 illustrates an expression level of Lrig-1 mRNA according to an embodiment of the present invention.

FIG. 5 illustrates an expression level of Lrig-1 mRNA according to an embodiment of the present invention.

FIG. 6 illustrates expression levels of Lrig-1, Lrig-2, and Lrig-3 mRNAs according to an embodiment of the present invention.

FIG. 7 illustrates results obtained by comparing expression levels of Lrig-1 protein in regulatory T cells and non-regulatory T cells according to an embodiment of the present invention.

FIG. 8 illustrates expression of the Lrig-1 protein on the surface of regulatory T cells according to an embodiment of the present invention.

FIG. 9 illustrates results obtained by analyzing binding capacity of antibodies (A7, C8, E7, G3, A8, B8, D9, and H6) to the Lrig-1 protein in an embodiment of the present invention.

FIG. 10 illustrates results obtained by analyzing the mechanism of regulating Lrig-1 protein-induced Stat3 phosphorylation, in regulatory T cells, of Lrig-1 protein-specific monoclonal antibodies (A7, C8, E7, G3, A8, B8, D9, and H6) in an embodiment of the present invention.

FIG. 11 illustrates an experimental design for cancer therapy using Lrig-1 protein-specific monoclonal antibodies (A8, B8, D9, and H6) in an embodiment of the present invention.

FIG. 12 illustrates cancer therapeutic effects obtained by using Lrig-1 protein-specific monoclonal antibodies (A8, B8, D9, and H6) in an embodiment of the present invention.

FIG. 13 illustrates results obtained by performing epitope mapping of 10 μg/ml of a monoclonal antibody (H6) to the Lrig-1 protein using a microarray in an embodiment of the present invention.

FIG. 14 illustrates results obtained by performing epitope mapping of 100 μg/ml of a monoclonal antibody (H6) to the Lrig-1 protein using a microarray in an embodiment of the present invention.

FIG. 15 illustrates results obtained by performing epitope mapping of a monoclonal antibody (H6) to the Lrig-1 protein using a microarray in an embodiment of the present invention.

### DETAILED DESCRIPTION OF INVENTION

The present invention relates to an epitope of leucine-rich and immunoglobulin-like domains 1 (Lrig-1) protein, or an antibody or antigen-binding fragment which specifically binds to the epitope.

According to an embodiment of the present invention, as the epitope of the Lrig-1 protein, there is provided an epitope including a polypeptide that consists of an amino acid sequence represented by Formula 1,

Lx¹Lx²x³N. [Formula 1]

In Formula 1, x¹ to x³ may each independently be a neutral amino acid, an acidic amino acid, a basic amino acid, or an aromatic amino acid. Here, the neutral amino acid may be glycine (G), alanine (A), valine (V), leucine (L), isoleucine (I), serine (S), or threonine (T); the acidic amino acid may be aspartic acid (D), glutamic acid (E), asparagine (N), or glutamine (Q); the basic amino acid may be lysine (K), arginine (R), or histidine (H); and the aromatic amino acid may be phenylalanine (F) or tyrosine (Y).

In an example of the present invention, x¹ to x³ may each independently be an amino acid selected from the group consisting of asparagine (N), aspartic acid (D), serine (S), tyrosine (Y), arginine (R), phenylalanine (F), lysine (K), histidine (H), leucine (L), valine (V), threonine (T), alanine (A), glutamine (Q), glutamic acid (E), and glycine (G).

In another example of the present invention, x¹ may be an amino acid selected from the group consisting of asparagine (N), phenylalanine (F), aspartic acid (D), lysine (K), histidine (H), valine (V), arginine (R), and threonine (T); x² may be an amino acid selected from the group consisting of serine (S), glutamine (Q), alanine (A), asparagine (N), glutamic acid (E), aspartic acid (D), phenylalanine (F), and glycine (G); and x³ may be an amino acid selected from the group consisting of tyrosine (Y), histidine (H), glycine (G), arginine (R), asparagine (N), leucine (L), lysine (K), and phenylalanine (F).

In yet another example of the present invention, x¹ to x³ may each independently be an amino acid selected from the group consisting of asparagine (N), aspartic acid (D), serine (S), tyrosine (Y), and arginine (R).

In still yet another example of the present invention, x¹ may be asparagine (N) or aspartic acid (D); x² may be serine (S) or asparagine (N); and x³ may be tyrosine (Y) or arginine (R).

In still yet another example of the present invention, the polypeptide consisting of the amino acid sequence represented by Formula 1 may be represented by, but is not limited to, any one amino acid sequence of SEQ ID NOs: 4 to 17.

According to another embodiment of the present invention, as the epitope of the Lrig-1 protein, there is provided an epitope including a polypeptide represented by any one amino acid sequence of SEQ ID NOs: 18 to 29 in Table 4.

According to yet another embodiment of the present invention, there is provided an antibody or antigen-binding fragment which specifically binds to the epitope of the present invention.

According to still yet another embodiment of the present invention, there is provided a pharmaceutical composition for preventing or treating cancer, comprising, as an active ingredient, the antibody or antigen-binding fragment provided by the present invention.

Hereinafter, the present invention will be described in more detail by way of examples. These examples are only for describing the present invention in more detail, and it will be apparent to those skilled in the art that according to the gist of the present invention, the scope of the present invention is not limited by these examples.

### Examples

### [Preparation Example 1] T Cell Subtype Cell Culture

In order to identify whether the Lrig-1 protein is expressed only in regulatory T cells (Treg) cells, the subsets of T cells, Th0, Th1, Th2, Th17, and iTreg, were prepared. The iTreg refers to cells whose differentiation has been artificially induced in a medium having the following composition, unlike nTreg which has been naturally isolated.

The subsets of the T cells were induced to differentiate into respective cells by first isolating naive T cells obtained from the spleen of mice, causing RPMI1640 (Invitrogen Gibco, Grand Island, NY) nutrient medium that contains 10% fetal bovine serum (FBS; HyClone, Logan, UT) to further contain the respective ingredients of Table 5 below, and performing 72-hour incubation in an incubator at 37° C., 5% CO₂.

TABLE 5

| Differentiated cell | Composition |
|---|---|
| Th0 | anti-CD3, anti-CD28 |
| Th1 | IL-12, anti-IL-4 antibody |
| Th2 | IL-4, anti-IFNβ |
| Th17 | IL-6, TGFβ, anti-IFNβ, anti-IL-4 |
| iTreg | IL-2, TGFβ |

### [Example 1] Structural Analysis of Lrig-1

A three-dimensional steric structure of the extracellular domain of the Lrig-1 protein was predicted to produce an antibody specific for the Lrig-1 protein, a surface protein of regulatory T cells.

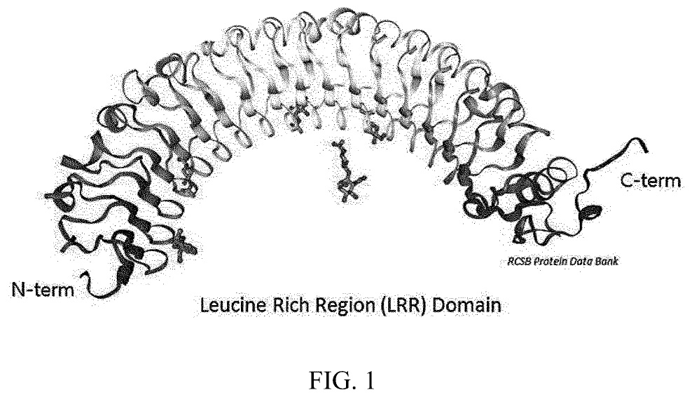

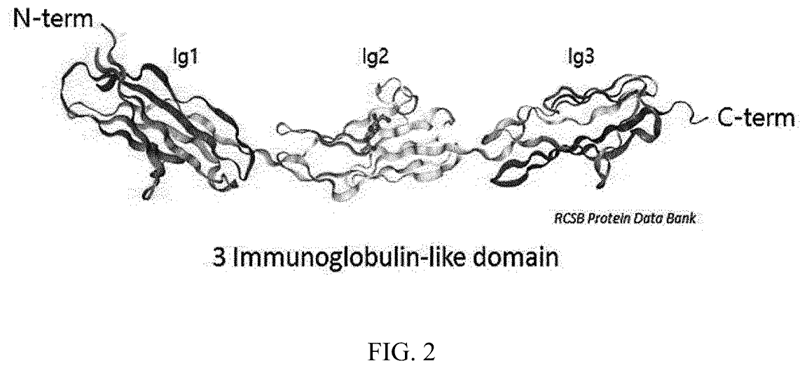

First, in order to predict base sequences of epitopes, tools of Uniprot (www.uniprot.org) and RCSB Protein Data Bank (www.rcsb.org/pdb) were used to predict a three-dimensional steric structure of the extracellular domain (ECD) of the Lrig-1 protein so that the structure of ECD is identified. Then, the results are illustrated in FIGS. 1 and 2.

As illustrated in FIG. 1, a total of 15 leucine-rich regions of LRR1 to LRR15 exist in the Lrig-LRR domain (amino acid sequence at positions 41 to 494) in the extracellular domain of the Lrig-1 protein. Each of the LRR domains is composed of 23 to 27 amino acids, with 3 to 5 leucine being present.

In addition, as illustrated in FIG. 2, three immunoglobulin-like domains exist in amino acid sequences at positions 494 to 781 of the Lrig-1 protein in the extracellular domain of the Lrig-1 protein.

### [Example 2] Identification of Specific Expression of Lrig-1 mRNA in Regulatory T Cells

Verification was made of whether the Lrig-1 protein can act as a biomarker specific for regulatory T cells.

For the verification, CD4⁺ T cells were isolated using magnet-activated cell sorting (MACS), through CD4 beads, from the spleen of mice. Subsequently, regulatory T (CD4⁺CD25⁺ T) cells and non-regulatory T (CD4⁺CD25⁻ T) cells were isolated with a fluorescence-activated cell sorter (FACS) using a CD25 antibody. For the respective cells and the cells differentiated in Preparation Example 1, mRNA was extracted using Trizol, and then gDNA was removed from genomic RNA using gDNA extraction kit (Qiagen) according to the protocol provided by the manufacturer. The gDNA-removed mRNA was synthesized into cDNA through the BDsprint cDNA Synthesis Kit (Clonetech).

Real-time polymerase chain reaction (RT PCR) was performed to quantitatively identify an expression level of Lrig-1 mRNA in the cDNA.

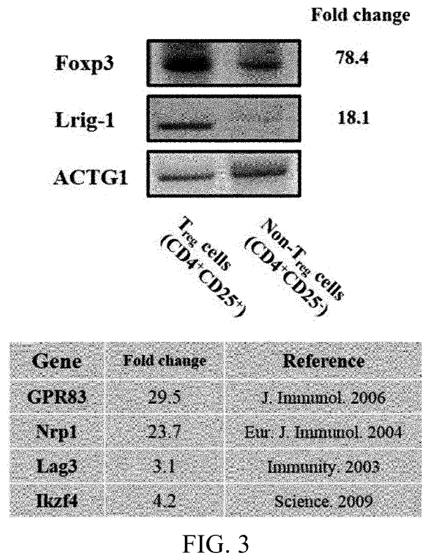

The real-time polymerase chain reaction was performed with primers shown in Table 6 below using SYBR Green (Molecular Probes) according to the protocol provided by the manufacturer under conditions of 40 cycles consisting of 95° C. for 3 minutes, 61° C. for 15 seconds, 72° C. for 30 seconds; and a relative gene expression level was calculated using the ΔCT method, and normalized using HPRT. The results are illustrated in FIGS. 3 to 6.

TABLE 6

| Primer | Sequence |
|---|---|
| Mouse Lrig-1 | Forward 5′ - GAC GGA ATT CAG TGA |
| | GGA GAA CCT - 3′ (SEQ ID NO: 46) |
| | Reverse 5′ - CAA CTG GTA GTG GCA |
| | GCT TGT AGG - 3′ (SEQ ID NO: 47) |
| Mouse Lrig-2 | Forward 5′ - TCA CAA GGA ACA TTG |
| | TCT GAA CCA - 3′ (SEQ ID NO: 48) |
| | Reverse 5′ - GCC TGA TCT AAC ACA |
| | TCC TCC TCA - 3′ (SEQ ID NO: 49) |
| Mouse Lrig-3 | Forward 5′ - CAG CAC CTT GAG CTG |
| | AAC AGA AAC - 3′ (SEQ ID NO: 50) |
| | Reverse 5′ - CCA GCC TTT GGT AAT |
| | CTC GGT TAG - 3′ (SEQ ID NO: 51) |
| Mouse FOXP3 | Forward 5′ - CTT TCA CCT ATC CCA |
| | CCC TTA TCC - 3′ (SEQ ID NO: 52) |
| | Reverse 5′ - ATT CAT CTA CGG TCC |
| | ACA CTG CTC - 3′ (SEQ ID NO: 53) |
| ACTG1 | Forward 5′ - GGC GTC ATG GTG GGC |
| | ATG GG - 3′ (SEQ ID NO: 54) |
| | Reverse 5′ - ATG GCG TGG GGA AGG |
| | GCG TA - 3′ (SEQ ID NO: 55) |

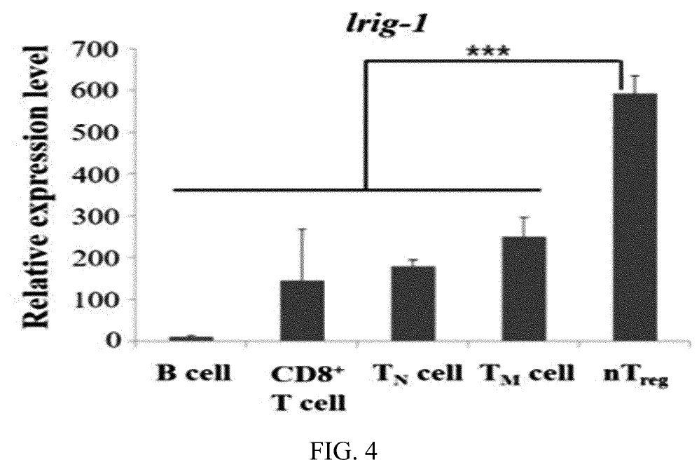

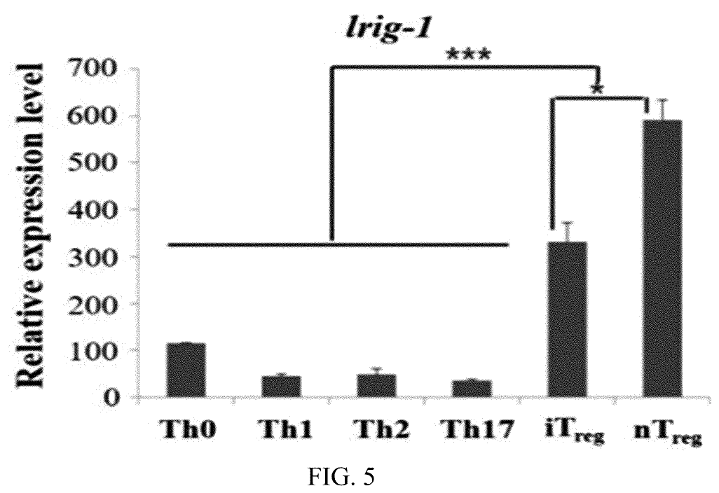

As illustrated in FIG. 3, it can be seen that the expression of Lrig-1 in regulatory T (CD4⁺CD25⁺ T) cells is 18.1 times higher than non-regulatory T (CD4⁺CD25⁻ T) cells. This was about 10 times higher expression level than Lag3 and Ikzf4, which are previously known markers for regulatory T cells. In addition, as illustrated in FIGS. 4 and 5, the expression of Lrig-1 mRNA was remarkably high in regulatory T cells as compared with other types of immune cells, and in particular, was remarkably high in naturally isolated regulatory T cells (nTreg) as compared with induced regulatory T cells (iTreg).

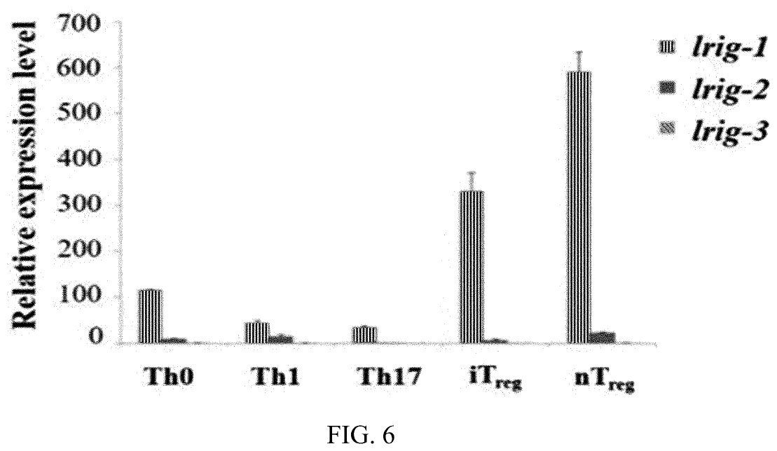

In addition, as illustrated in FIG. 6, the expression of Lrig-1 was the highest among Lrig-1, Lrig-2, and Lrig-3 which correspond to the Lrig family.

From the above results, it can be seen that the Lrig-1 protein according to the present invention is specifically expressed in regulatory T cells, in particular, naturally-occurring regulatory T cells.

### [Example 3] Identification of Specific Expression of Lrig-1 Protein in Regulatory T Cells

It was identified whether the Lrig-1 protein expressed from Lrig-1 mRNA is specifically expressed only in regulatory T cells.

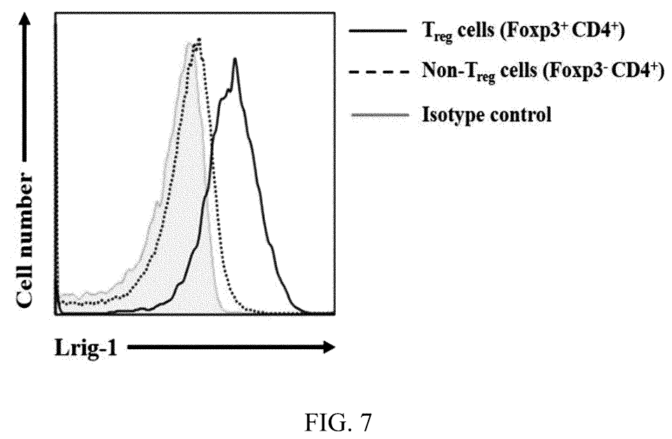

Using FOXP3-RFP-knocked-in mice, the FOXP3-RFP obtained by coupling red fluorescence protein (RFP) to FOXP3 promoter, which is a transcription factor specific for regulatory T cells, CD4⁺ T cells were isolated using magnet-activated cell sorting (MACS), through CD4 beads, from the spleen of the mice. Subsequently, using RFP protein, regulatory T (CD4⁺RFP⁺ T) cells and non-regulatory T (CD4⁺RFP⁻ T) cells were obtained by performing isolation through a fluorescence-activated cell sorter (FACS). The respective cells were stained with the purchased Lrig-1 antibody and a negative control was stained with an isotype-matched control antibody, to measure an expression level of Lrig-1 with the fluorescence-activated cell sorter. The results are illustrated in FIG. 7.

As illustrated in FIG. 7, the non-regulatory T cells indicated by a dotted line showed almost the same expression level of Lrig-1 as the negative control, whereas there were a large number of cells with high expression level of Lrig-1 in the regulatory T cells.

From the above results, it can be seen that the Lrig-1 protein according to the present invention is specifically expressed in regulatory T cells.

### [Example 4] Identification of Specific Expression of Lrig-1 Protein on Surface of Regulatory T Cells

From the viewpoint that in order to be a target of cell therapy, the Lrig-1 protein must be expressed on the surface of regulatory T cells, which in turn allows a more effective target therapy, it was identified whether the Lrig-1 protein is expressed on the surface of the regulatory T cells.

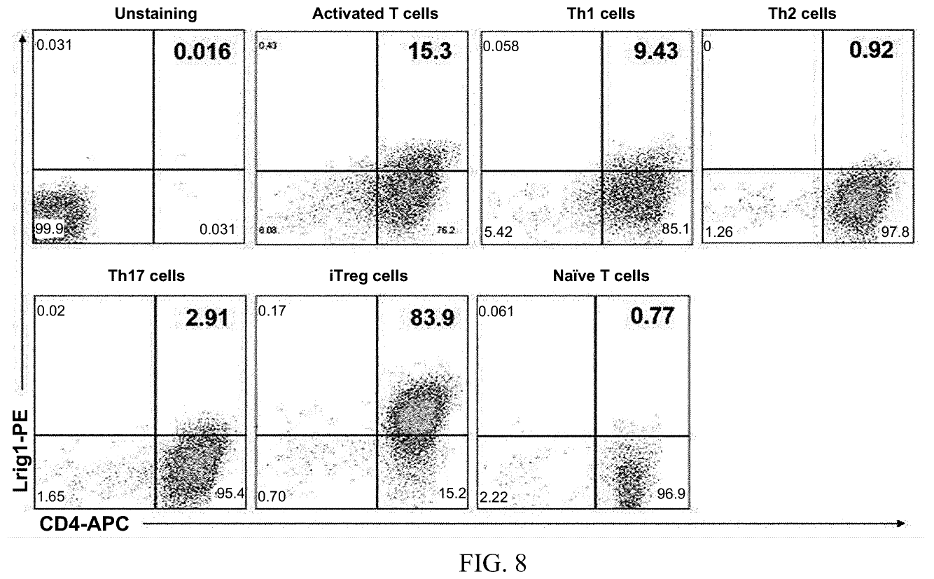

The respective differentiated T cell subsets of Preparation Example 1 were stained with anti-CD4-APC and anti-Lrig-1-PE antibodies, and expression levels of Lrig-1 were measured at the respective cell surfaces using a fluorescence-activated cell sorter (FACS). The results are illustrated in FIG. 8.

As illustrated in FIG. 8, Lrig-1 was expressed in an amount of 0.77 to 15.3 in activated T cells, Th1 cells, Th2 cells, Th17 cells, and naive T cells, whereas Lrig-1 was expressed as high as 83.9 in differentiation-induced T cells (iTreg cells).

From the above results, it can be seen that the Lrig-1 protein according to the present invention is not only specifically expressed in regulatory T (Treg) cells, but also is, in particular, expressed at a higher level on the surface of the Treg cells.

### [Production Examples 1 to 8] Production of Monoclonal Antibodies Specific for Lrig-1 Protein

Antibodies specific for the Lrig-1 protein according to the present invention were produced. The present antibodies were not produced by specifying a certain epitope, but were produced as antibodies capable of binding to any site on the Lrig-1 protein.

In order to produce the antibodies, cells expressing the Lrig-1 protein were produced. More specifically, a DNA fragment corresponding to SEQ ID NO: 2 and pcDNA (hygro) were cleaved with a cleavage enzyme, incubated at 37° C., and ligated to produce pcDNA into which a DNA sequence of the Lrig-1 protein is inserted. The thus produced pcDNA into which SEQ ID NO: 2 is inserted was introduced, through transfection, into L cells, so that the Lrig-1 protein is allowed to be expressed on the surface of the L cells.

Light and heavy chain amino acid sequences capable of binding to Lrig-1 expressed on the cell surface were selected from the Human scFv library so that a total of eight heavy and light chains were selected.

The selected heavy and light chain amino acid sequences were fused with the mlgG2a Fc region, to produce monoclonal antibodies. The sequences of the monoclonal antibodies are shown in Table 7 below.

TABLE 7

| Classifi- | | | | Sequence |
|---|---|---|---|---|
| cation | Clone | Location | Amino acid sequence | information |
| Production | A7 | Heavy | EVQLLESGGGLVQPGGSLRLSCAASGFTFSGYDMSWVRQAPGKGLEWVSLIYPDS | SEQ ID NO: 30 |
| Example 1 | clone | chain | GNKYYADSVKGRFTISRDNSKNTLYLQMNSLRAEDTAVYYCARDAGLSWAGAFD | |
| | | | YWGQGTLVTVSSTTAPSVYPLAPVCGDTTGSSVTLGCLVKGYFPEPVTLTWNSGSL | |
| | | | SSGVHTFPAVLQSDLYTLSSSVTVTSSTWPSQSITCNVAHPASSTKVDKKIEPRGPTI | |
| | | | KPCPPCKCPAPNLLGGPSVFIFPPKIKDVLMISLSPIVTCVVVDVSEDDPDVQISWFV | |
| | | | NNVEVHTAQTQTHREDYNSTLRVVSALPIQHQDWMSGKEFKCKVNNKDLPAPIER | |
| | | | TISKPKGSVRAPQVYVLPPPEEEMTKKQVTLTCMVTDFMPEDIYVEWTNNGKTEL | |
| | | | NYKNTEPVLDSDGSYFMYSKLRVEKKNWVERNSYSCSVVHEGLHNHHTTKSFSR | |
| | | | TPGK | |
| | | Light | QSVLTQPPSASGTPGQRVTISCSGSSSNIGSNYVTWYQQLPGTAPKLLIYSDSHRPSG | SEQ ID NO: 31 |
| | | chain | VPDRFSGSKSGTSASLAISGLQSEDEADYYCGSWDYSLSAYVFGGGTKLTVLRTVA | |
| | | | APTVSIFPPSSEQLTSGGASVVCFLNNFYPKDINVKWKIDGSERQNGVLNSWTDQD | |
| | | | SKDSTYSMSSTLTLTKDEYERHNSYTCEATHKTSTSPIVKSFNRNEC | |
| Production | C8 clone | Heavy | EVQLLESGGGLVQPGGSLRLSCAASGFTFSNYYMSWVRQAPGKGLEWVSGISPGD | SEQ ID NO: 32 |
| Example 2 | | chain | SSTYYADSVKGRFTISRDNSKNTLYLQMNSLRAEDTAVYYCAKGLYSNPNEPFDY | |
| | | | WGQGTLVTVSSTTAPSVYPLAPVCGDTTGSSVTLGCLVKGYFPEPVTLTWNSGSLS | |
| | | | SGVHTFPAVLQSDLYTLSSSVTVTSSTWPSQSITCNVAHPASSTKVDKKIEPRGPTIK | |
| | | | PCPPCKCPAPNLLGGPSVFIFPPKIKDVLMISLSPIVTCVVVDVSEDDPDVQISWFVN | |
| | | | NVEVHTAQTQTHREDYNSTLRVVSALPIQHQDWMSGKEFKCKVNNKDLPAPIERTI | |
| | | | SKPKGSVRAPQVYVLPPPEEEMTKKQVTLTCMVTDFMPEDIYVEWINNGKTELN | |
| | | | YKNTEPVLDSDGSYFMYSKLRVEKKNWVERNSYSCSVVHEGLHNHHTTKSFSRTP | |
| | | | GK | |
| | | Light | QSVLTQPPSASGTPGQRVTISCTGSSSNIGSNYVSWYQQLPGTAPKLLIYDDSQRPS | SEQ ID NO: 33 |
| | | chain | GVPDRFSGSKSGTSASLAISGLRSEDEADYYCGTWDYSLNGYVFGGGTKLTVLRT | |
| | | | VAAPTVSIFPPSSEQLTSGGASVVCFLNNFYPKDINVKWKIDGSERQNGVLNSWTD | |
| | | | QDSKDSTYSMSSTLTLTKDEYERHNSYTCEATHKTSTSPIVKSFNRNEC | |
| Production | E7 clone | Heavy | EVQLLESGGGLVQPGGSLRLSCAASGFTFSSYDMSWVRQAPGKGLEWVSGISPDG | SEQ ID NO: 34 |
| Example 3 | | chain | SNIYYADSVKGRFTISRDNSKNTLYLQMNSLRAEDTAVYYCAKVGLRCRYEACSYA | |
| | | | YGMDVWGQGTLVTVSSTTAPSVYPLAPVCGDTTGSSVTLGCLVKGYFPEPVTLTW | |
| | | | NSGSLSSGVHTFPAVLQSDLYTLSSSVTVTSSTWPSQSITCNVAHPASSTKVDKKIEP | |
| | | | RGPTIKPCPPCKCPAPNLLGGPSVFIFPPKIKDVLMISLSPIVTCVVVDVSEDDPDVQI | |
| | | | SWFVNNVEVHTAQTQTHREDYNSTLRVVSALPIQHQDWMSGKEFKCKVNNKDLP | |
| | | | APIERTISKPKGSVRAPQVYVLPPPEEEMTKKQVTLTCMVTDFMPEDIYVEWTNNG | |
| | | | KTELNYKNTEPVLDSDGSYFMYSKLRVEKKNWVERNSYSCSVVHEGLHNHHTTK | |
| | | | SFSRTPGK | |
| | | Light | QSVLTQPPSASGTPGQRVTISCSGSSSNIGSNYVSWYQQLPGTAPKLLIYSDSHRPSG | SEQ ID NO: 35 |
| | | chain | VPDRFSGSKSGTSASLAISGLRSEDEADYYCATWDSSLNGYVFGGGTKLTVLRTVA | |
| | | | APTVSIFPPSSEQLTSGGASVVCFLNNFYPKDINVKWKIDGSERQNGVLNSWTDQD | |
| | | | SKDSTYSMSSTLTLTKDEYERHNSYTCEATHKTSTSPIVKSFNRNEC | |
| Production | G3 | Heavy | EVQLLESGGGLVQPGGSLRLSCAASGFTFSNYDMSWVRQAPGKGLEWVSSISPSSG | SEQ ID NO: 36 |
| Example 4 | clone | chain | SIYYADSVKGRFTISRDNSKNTLYLQMNSLRAEDTAVYYCAKDLDAFWRPSFDYW | |
| | | | GQGTLVTVSSTTAPSVYPLAPVCGDTTGSSVTLGCLVKGYFPEPVTLTWNSGSLSSG | |
| | | | VHTFPAVLQSDLYTLSSSVTVTSSTWPSQSITCNVAHPASSTKVDKKIEPRGPTIKPCP | |
| | | | PCKCPAPNLLGGPSVFIFPPKIKDVLMISLSPIVTCVVVDVSEDDPDVQISWFVNNVE | |
| | | | VHTAQTQTHREDYNSTLRVVSALPIQHQDWMSGKEFKCKVNNKDLPAPIERTISKP | |
| | | | KGSVRAPQVYVLPPPEEEMTKKQVTLTCMVTDFMPEDIYVEWTNNGKTELNYKN | |
| | | | TEPVLDSDGSYFMYSKLRVEKKNWVERNSYSCSVVHEGLHNHHTTKSFSRTPGK | |
| | | Light | QSVLTQPPSASGTPGQRVTISCTGSSSNIGNNNVNWYQQLPGTAPKLLIYSDSHRPS | SEQ ID NO: 37 |
| | | chain | GVPDRFSGSKSGTSASLAISGLRSEDEADYYCGSWDDSLSAYVFGGGTKLTVLRTV | |
| | | | AAPTVSIFPPSSEQLTSGGASVVCFLNNFYPKDINVKWKIDGSERQNGVLNSWTDQ | |
| | | | DSKDSTYSMSSTLTLTKDEYERHNSYTCEATHKTSTSPIVKSFNRNEC | |
| Production | A8 | Heavy | EVQLLESGGGLVQPGGSLRLSCAASGFTFSDYDMSWVRQVPGKGLEWVSWISHG | SEQ ID NO: 38 |
| Example 5 | clone | chain | GGSIYYADSVKGRFTISRDNSKNTLYLQMNSLRAEDTAVYYCARGLGLCKTGLCY | |
| | | | YYDAMDVWGQGTLVTVSSTTAPSVYPLAPVCGDTTGSSVTLGCLVKGYFPEPVTL | |
| | | | TWNSGSLSSGVHTFPAVLQSDLYTLSSSVTVTSSTWPSQSITCNVAHPASSTKVDKKI | |
| | | | EPRGPTIKPCPPCKCPAPNLLGGPSVFIFPPKIKDVLMISLSPIVTCVVVDVSEDDPDV | |
| | | | QISWFVNNVEVHTAQTQTHREDYNSTLRVVSALPIQHQDWMSGKEFKCKVNNKD | |
| | | | LPAPIERTISKPKGSVRAPQVYVLPPPEEEMTKKQVTLTCMVTDFMPEDIYVEWTN | |
| | | | NGKTELNYKNTEPVLDSDGSYFMYSKLRVEKKNWVERNSYSCSVVHEGLHNHHT | |
| | | | TKSFSRTPGK | |
| | | Light | QSVLTQPPSASGTPGQRVTISCTGSSSNIGNNSVTWYQQLPGTAPKLLIYADNNRPS | SEQ ID NO: 39 |
| | | chain | GVPDRFSGSKSGTSASLAISGLRSEDEADYYCAAWDSSLSAYVFGGGTKLTVLRTV | |
| | | | AAPTVSIFPPSSEQLTSGGASVVCFLNNFYPKDINVKWKIDGSERQNGVLNSWTDQ | |
| | | | DSKDSTYSMSSTLTLTKDEYERHNSYTCEATHKTSTSPIVKSFNRNEC | |
| Production | B8 clone | Heavy | EVQLLESGGGLVQPGGSLRLSCAASGFTFSDYYMSWVRQAPGKGLEWVSGISHDS | SEQ ID NO: 40 |
| Example 6 | | chain | GSKYYADSVKGRFTISRDNSKNTLYLQMNSLRAEDTAVYYCARHWTTFDYWGQG | |
| | | | TLVTVSSTTAPSVYPLAPVCGDTTGSSVTLGCLVKGYFPEPVTLTWNSGSLSSGVHT | |
| | | | FPAVLQSDLYTLSSSVTVTSSTWPSQSITCNVAHPASSTKVDKKIEPRGPTIKPCPPCK | |
| | | | CPAPNLLGGPSVFIFPPKIKDVLMISLSPIVTCVVVDVSEDDPDVQISWFVNNVEVH | |
| | | | TAQTQTHREDYNSTLRVVSALPIQHQDWMSGKEFKCKVNNKDLPAPIERTISKPKG | |
| | | | SVRAPQVYVLPPPEEEMTKKQVTLTCMVTDFMPEDIYVEWINNGKTELNYKNTEP | |
| | | | VLDSDGSYFMYSKLRVEKKNWVERNSYSCSVVHEGLHNHHTTKSFSRTPGK | |
| | | Light | QSVLTQPPSASGTPGQRVTISCSGSSSNIGSNNVTWYQQLPGTAPKLLIYANSNRPSG | SEQ ID NO: 41 |
| | | chain | VPDRFSGSKSGTSASLAISGLRSEDEADYYCGAWDYSLSAYVFGGGTKLTVLRTVA | |
| | | | APTVSIFPPSSEQLTSGGASVVCFLNNFYPKDINVKWKIDGSERQNGVLNSWTDQD | |
| | | | SKDSTYSMSSTLTLTKDEYERHNSYTCEATHKTSTSPIVKSFNRNEC | |
| Production | D9 | Heavy | EVQLLESGGGLVQPGGSLRLSCAASGFTFSNYAMSWVRQAPGKGLEWVSAIYPGG | SEQ ID NO: 42 |
| Example 7 | clone | chain | GSIYYADSVKGRFTISRDNSKNTLYLQMNSLRAEDTAVYYCARDILPCPWGRCYYD | |
| | | | YAMDVWGQGTLVTVSSTTAPSVYPLAPVCGDTTGSSVTLGCLVKGYFPEPVTLTW | |
| | | | NSGSLSSGVHTFPAVLQSDLYTLSSSVTVTSSTWPSQSITCNVAHPASSTKVDKKIEP | |
| | | | RGPTIKPCPPCKCPAPNLLGGPSVFIFPPKIKDVLMISLSPIVTCVVVDVSEDDPDVQI | |
| | | | SWFVNNVEVHTAQTQTHREDYNSTLRVVSALPIQHQDWMSGKEFKCKVNNKDLP | |
| | | | APIERTISKPKGSVRAPQVYVLPPPEEEMTKKQVTLTCMVTDFMPEDIYVEWTNNG | |
| | | | KTELNYKNTEPVLDSDGSYFMYSKLRVEKKNWVERNSYSCSVVHEGLHNHHTTK | |
| | | | SFSRTPGK | |
| | | Light | QSVLTQPPSASGTPGQRVTISCSDSSSNIGSNTVSWYQQLPGTAPKLLIYADNNRPSG | SEQ ID NO: 43 |
| | | chain | VPDRFSGSKSGTSASLAISGLRSEDEADYYCGTWDYSLSGYVFGGGTKLTVLRTVA | |
| | | | APTVSIFPPSSEQLTSGGASVVCFLNNFYPKDINVKWKIDGSERQNGVLNSWTDQD | |
| | | | SKDSTYSMSSTLTLTKDEYERHNSYTCEATHKTSTSPIVKSFNRNEC | |
| Production | H6 | Heavy | EVQLLESGGGLVQPGGSLRLSCAASGFTFSNYAMSWVRQAPGKGLEWVSVISHGG | SEQ ID NO: 44 |
| Example 8 | clone | chain | GSTYYADSVKGRFTISRDNSKNTLYLQMNSLRAEDTAVYYCARVISNCHLGVCYYS | |
| | | | NGMDVWGQGTLVTVSSTTAPSVYPLAPVCGDTTGSSVTLGCLVKGYFPEPVTLTW | |
| | | | NSGSLSSGVHTFPAVLQSDLYTLSSSVTVTSSTWPSQSITCNVAHPASSTKVDKKIEP | |
| | | | RGPTIKPCPPCKCPAPNLLGGPSVFIFPPKIKDVLMISLSPIVTCVVVDVSEDDPDVQI | |
| | | | SWFVNNVEVHTAQTQTHREDYNSTLRVVSALPIQHQDWMSGKEFKCKVNNKDLP | |
| | | | APIERTISKPKGSVRAPQVYVLPPPEEEMTKKQVTLTCMVTDFMPEDIYVEWTNNG | |
| | | | KTELNYKNTEPVLDSDGSYFMYSKLRVEKKNWVERNSYSCSVVHEGLHNHHTTK | |
| | | | SFSRTPGK | |
| | | Light | QSVLTQPPSASGTPGQRVTISCSGSSSNIGNNDVYWYQQLPGTAPKLLIYSDSQRPS | SEQ ID NO: 45 |
| | | chain | GVPDRFSGSKSGTSASLAISGLRSEDEADYYCGTWDYSLSGYVFGGGTKLTVLRTV | |
| | | | AAPTVSIFPPSSEQLTSGGASVVCFLNNFYPKDINVKWKIDGSERQNGVLNSWTDQ | |
| | | | DSKDSTYSMSSTLTLTKDEYERHNSYTCEATHKTSTSPIVKSFNRNEC | |

### [Example 5] Evaluation of Binding Capacity of Antibodies According to Present Invention to Lrig-1 Protein

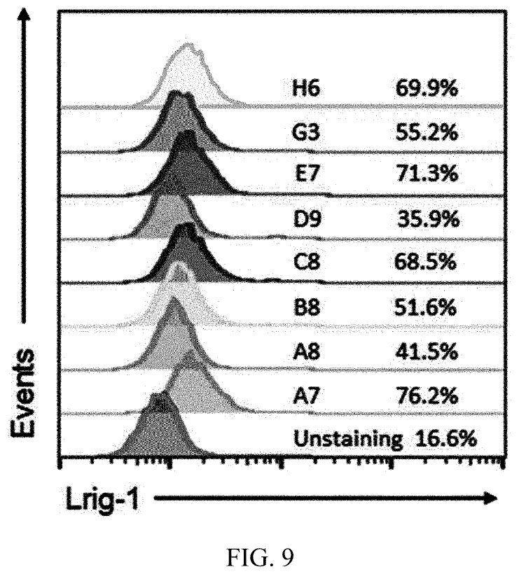

In order to identify whether the monoclonal antibodies according to the present invention produced in Production Examples 1 to 8 well recognize Lrig-1, each of the antibodies of Production Examples 1 to 8 was bound to L cells that stably express Lrig-1. Then, a secondary antibody which is conjugated with eFlour 670 and is capable of recognizing mouse antibodies was added thereto, and then binding capacity of the monoclonal antibodies to the Lrig-1 protein was analyzed using FACS. The results are illustrated in FIG. 9.

As illustrated in FIG. 9, it was found that all Lrig-1 protein-specific monoclonal antibodies (A7, A8, B8, C8, D9, E7, G3, and H6) according to the present invention effectively recognize and bind to the Lrig-1 protein present on the surface of L cells.

### [Example 6] Regulation of Signal Transduction Pathway in Regulatory T Cells, by Antibodies According to Present Invention

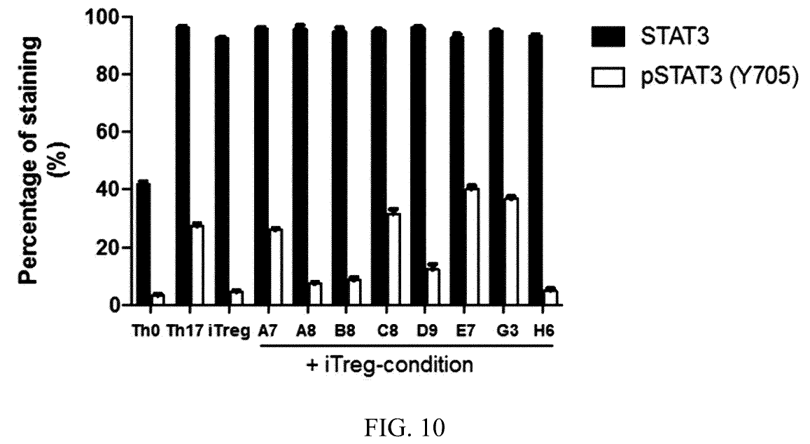

In order to analyze how the monoclonal antibodies according to the present invention produced in Production Examples 1 to 8 affect the signal transduction pathway in regulatory T cells through the Lrig-1 protein, Lrig-1 present on the surface of the regulatory T cells was stimulated by treating the regulatory T cells with the antibodies of Production Examples 1 to 8, and then a level of tyrosine phosphorylation of Stat3 protein present in the stimulated regulatory T cells was analyzed through phosphotyrosine immunoblot. The results are illustrated in FIG. 10.

As illustrated in FIG. 10, it was found that the Lrig-1 protein-specific monoclonal antibodies (A7, C8, E7, and G3) according to the present invention increase phosphorylation of Stat3 to the same level as Th17 cells. On the other hand, it was found that the Lrig-1 protein-specific monoclonal antibodies (A8, B8, D9, and H6) according to the present invention continue to maintain and decrease phosphorylation of Stat3 at the same level as iTreg cells.

### [Example 7] Cancer Therapeutic Effects of A8, B8, D9, and H6 Antibodies

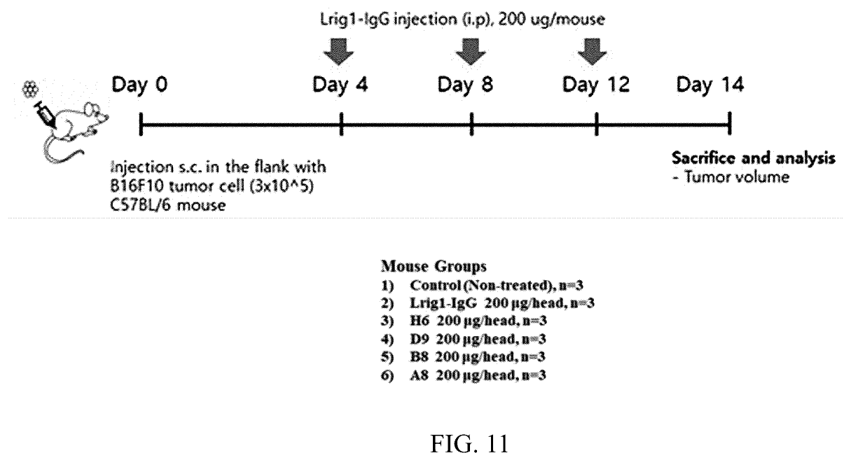

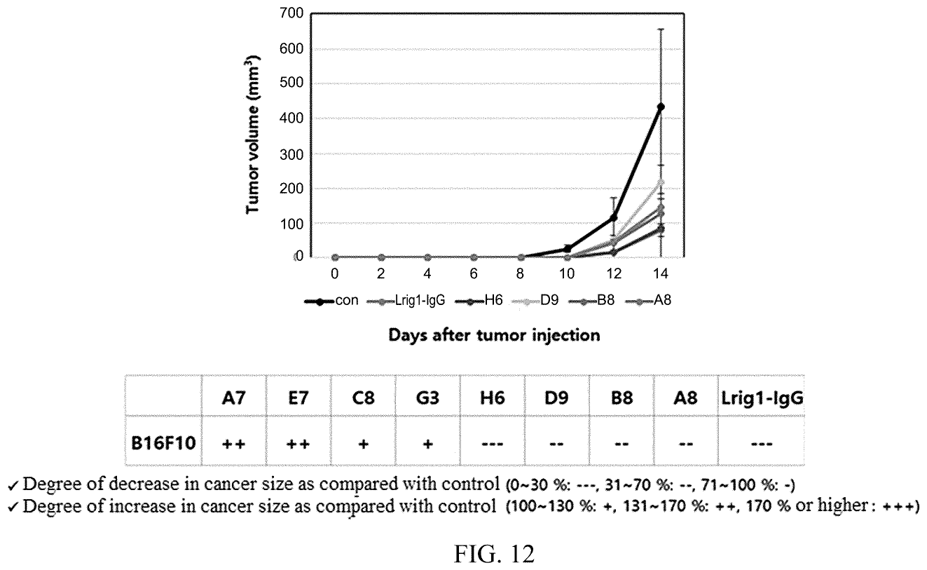

In order to identify therapeutic effects, on solid cancer, of the monoclonal antibodies (A8, B8, D9, and H6) according to the present invention, which had been produced in Production Examples 5 to 8, as illustrated in FIG. 11, B16F10 melanoma cells were subcutaneously injected into the dorsal area of mice in an amount of 3′10⁵ cells, and then the antibodies of Production Examples 5 to 8 were intraperitoneally injected into the mice in an amount of 200 μg on days 4, 8, and 12. After transplantation of the melanoma cells, changes in tumor volume over time were measured and the results are illustrated in FIG. 12.

As illustrated in FIG. 12, it was found that remarkably decreased melanoma tumor sizes are observed in a case of being treated with the Lrig-1 protein-specific monoclonal antibodies (A8, B8, D9, and H6) according to the present invention, as compared with a negative control having not received antibody treatment.

From these results, it can be seen that the Lrig-1 protein-specific monoclonal antibodies according to the present invention suppress growth of various solid cancer cells, thereby effectively preventing, ameliorating, or treating such cancers.

### [Example 8] Determination of Epitopes in Lrig-1 Protein According to Present Invention

In order to discover epitopes that bind to the H6 antibody of Production Example 8, in which the epitopes are present in respective LRR domains and Ig-like domains in the Lrig-1 protein present on the surface of regulatory T cells, the following experiment was performed.

### 1. Experimental Preparation

### (1) Microarray

The Lrig-1 protein was elongated by GSGSGSG linkers at the C- and N-termini thereof to avoid truncation of peptides. The elongated antigenic sequence was translated into 15 amino acids with a peptide-peptide overlap of 14 amino acids. The resulting Lrig-1 peptide microarrays contained 1,091 different peptides printed in duplicates and framed by additional HA (YPYDVPDYAG, 102 spots) control peptides.

### (2) Experimental Material

Sample: H6 monoclonal antibody of Production Example 8
Washing buffer: PBS, pH 7.4 with 0.05% Tween 20 (3 times, 10 seconds after each incubation)
Blocking buffer: Rockland blocking buffer MB-070 (30 minutes before first assay)
Incubation buffer: wash buffer with 10% blocking buffer
Assay conditions: antibody concentration in incubation buffer being adjusted to 1 μg/ml, 10 μg/ml, and 100 μg/ml; and incubation being performed at 4° C. for 16 hours, and stirring being performed at 140 rpm
Secondary antibody: goat anti-human IgG (H+L) DyLight680 (0.2 μg/ml); staining being performed in incubation buffer at room temperature for 45 minutes
Control antibody: mouse monoclonal anti-HA (12CA5) DyLight800 (0.5 μg/ml); staining being performed in incubation buffer at room temperature for 45 minutes
Scanner: LI-COR Odyssey Imaging System; scanning offset 0.65 mm, resolution 21 μm, scanning intensity of 7/7 (red=700 nm/green=800 nm)

### 2. Experimental Method

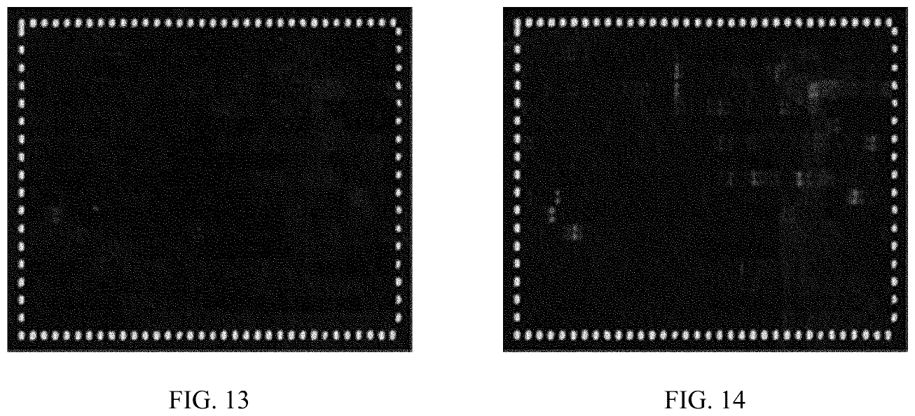

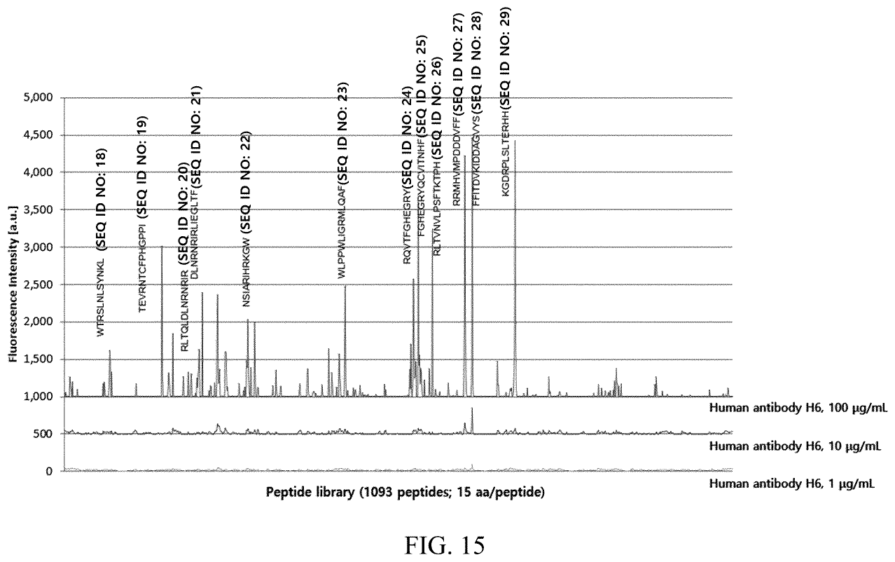

Pre-staining of a Lrig-1 peptide microarray copy using secondary and control antibodies in incubation buffer was performed to identify whether they can interact with antigen-derived peptides that can interfere with the main assays. Subsequent incubation of other Lrig-1 peptide microarray copies with the H6 monoclonal antibody of Production Example 8 (1 μg/ml, 10 μg/ml, and 100 μg/ml) in incubation buffer was followed by staining with secondary and control antibodies. Read-out was performed at a scanning intensity of 7/7 (red/green) using the LI-COR Odyssey Imaging System. Quantification of spot intensities and peptide annotation were based on the 16-bit gray scale tiff files. Microarray image analysis was done with PepSlide® Analyzer. The results are illustrated in FIGS. 13 to 15, and the analyzed epitope sequences are shown in Table 8 below.

TABLE 8

| SEQ ID NO | Sequence information |
|---|---|
| SEQ ID NO: 18 | WTRSLNLSYNKL |
| SEQ ID NO: 19 | TEVRNTCFPHGPPI |
| SEQ ID NO: 20 | RLTQLDLNRNRIR |
| SEQ ID NO: 21 | DLNRNRIRLIEGLTF |
| SEQ ID NO: 22 | NSIARIHRKGW |
| SEQ ID NO: 23 | WLPPWLIGRMLQAF |
| SEQ ID NO: 24 | RQVTFGHEGRY |
| SEQ ID NO: 25 | FGHEGRYQCVITNHF |
| SEQ ID NO: 26 | RLTVNVLPSFTKTPH |
| SEQ ID NO: 27 | RRMHVMPDDDVFF |
| SEQ ID NO: 28 | FFITDVKIDDAGVYS |
| SEQ ID NO: 29 | KGDRPLSLIERHH |

### 3. Experimental Results

As illustrated in FIGS. 13 to 15, it was found that the monoclonal antibody (H6), which specifically binds to Lrig-1 protein present on regulatory T cells and thus effectively treats cancer, specifically binds to an epitope represented by any one amino acid sequence of SEQ ID NOs: 18 to 29 in the Lrig-1 protein and thus exerts such a function. Furthermore, it was found that the epitopes represented by SEQ ID NOs: 18 and 20 have a leucine-rich repeat in common, and specifically, these epitopes contain a consensus sequence, which may be represented by the amino acid sequence of Formula 1 of the present invention, at the fifth amino acid position from the N-terminus.

Although the present invention has been described in detail above, the scope of the present invention is not limited thereto. It will be obvious to those skilled in the art that various modifications and changes can be made without departing from the technical spirit of the present invention described in the claims.

### Industrial Availability

The present invention relates to an epitope of leucine-rich and immunoglobulin-like domains 1 (Lrig-1) protein, which is an antigen present on the surface of regulatory T cells, and an antibody or antigen-binding fragment specifically binding thereto.

What is claimed is:

1. A method for preventing or treating cancer by decreasing phosphorylation of STAT3, the method comprising administering, to an individual, an antibody or antigen-binding fragment thereof that specifically binds to the Lrig-1 protein, the antibody or the antigen-binding fragment thereof comprising at least one polypeptide having an amino acid sequence of any of SEQ ID NOs: 4 to 29.

2. The method according to claim 1, wherein the antibody or the antigen-binding fragment thereof comprises:
an antibody heavy chain variable region (VH) comprising the amino acid sequence of SEQ ID No: 38, 40, 42, or 44; and
an antibody light chain variable region (VL) comprising the amino acid sequence of SEQ ID No: 39, 41, 43, or 45.

3. The method according to claim 1, wherein the cancer is gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, or lung cancer.

4. The method according to claim 2, wherein the cancer is gastric cancer, liver cancer, glioblastoma, ovarian cancer, colorectal cancer, head and neck cancer, bladder cancer, renal cell cancer, breast cancer, metastatic cancer, prostate cancer, pancreatic cancer, melanoma, or lung cancer.

5. The method according to claim 1, wherein the antibody or the antigen-binding fragment thereof comprises:
an antibody heavy chain variable region (VH) comprising the amino acid sequence of SEQ ID No: 38; and
an antibody light chain variable region (VL) comprising the amino acid sequence of SEQ ID No: 39.

6. The method according to claim 1, wherein the antibody or the antigen-binding fragment thereof comprises:
an antibody heavy chain variable region (VH) comprising the amino acid sequence of SEQ ID No: 40; and
an antibody light chain variable region (VL) comprising the amino acid sequence of SEQ ID No: 41.

7. The method according to claim 1, wherein the antibody or the antigen-binding fragment thereof comprises:
an antibody heavy chain variable region (VH) comprising the amino acid sequence of SEQ ID No: 42; and
an antibody light chain variable region (VL) comprising the amino acid sequence of SEQ ID No: 43.

8. The method according to claim 1, wherein the antibody or the antigen-binding fragment thereof comprises:
an antibody heavy chain variable region (VH) comprising the amino acid sequence of SEQ ID No: 44; and
an antibody light chain variable region (VL) comprising the amino acid sequence of SEQ ID No: 45.
